(19) United States
(12) Patent Application Publication
(10) Pub. No.: US 2013/0149243 A1
(43) Pub. Date: Jun. 13, 2013

(54) METHOD FOR PRODUCTION OF F-18 LABELED GLUTAMIC ACID DERIVATIVES

(71) Applicants: Mathias Berndt, Berlin (DE); Heribert Schmitt-Willich, Berlin (DE); Matthias Friebe, Berlin (DE); Keith Graham, Berlin (DE); Thomas Brumby, Berlin (DE); Christina Hultsch, Berlin (DE); Hans-Jürgen Wester, Ilmmunster (DE); Franziska Wagner, Hergolding (DE)

(72) Inventors: Mathias Berndt, Berlin (DE); Heribert Schmitt-Willich, Berlin (DE); Matthias Friebe, Berlin (DE); Keith Graham, Berlin (DE); Thomas Brumby, Berlin (DE); Christina Hultsch, Berlin (DE); Hans-Jürgen Wester, Ilmmunster (DE); Franziska Wagner, Hergolding (DE)

(73) Assignee: BAYER INTELLECTUAL PROPERTY GMBH, Monheim (DE)

(21) Appl. No.: 13/510,365

(22) PCT Filed: Nov. 6, 2010
(86) PCT No.: PCT/EP10/06766

§ 371 Date: Feb. 28, 2013

(30) Foreign Application Priority Data
Nov. 17, 2009 (EP) 09075508.3

(51) Int. Cl. C07C 227/20; A61K 51/04; C07C 309/73; C07C 309/66; C07C 271/22

(52) U.S. Cl. CPC C07C 227/20; C07C 309/66; C07C 271/22; C07C 309/73; A61K 51/0406
USPC 424/1.89; 562/571; 558/49; 560/157; 422/527

(57) ABSTRACT

This invention relates to methods, which provide access to F-18 labeled glutamic acid derivatives.

### FIELD OF INVENTION

This invention relates to methods, which provide access to F-18 labeled glutamic acid derivatives.

### BACKGROUND

Over the last few years, in vivo scanning using Positron Emission Tomography (PET) has increased. PET is both a medical and research tool. It is used in a variety of medical applications, including imaging of the brain, tumors, and components of cardiovascular system. Radiotracer consisting of a radionuclide bound to a biologically active compound is used for in vivo imaging of disorders.

The radionuclides used in PET scanning are typically isotopes with short half lives such as C-11 (˜20 min), N-13 (˜10 min), O-15 (˜2 min), Ga-68 (˜68 min) or F-18 (˜110 min). Due to their short half lives, the radionuclides must be produced in a cyclotron (or generator) which is not too far away in delivery-time from the PET scanner. These radionuclides are incorporated into biologically active compounds or biomolecules that have the function to vehicle the radionuclide into the body through the targeted site e.g. tumor.

F-18 labeled compounds are gaining importance due to its availability as well as development of methods for labeling biomolecules. It has been shown that some compounds labeled with F-18, produce images of high quality. Additionally, the longer lifetime of F-18 would permit longer imaging times and allows preparation of radiotracer batches for multiple patients and delivery of the tracer to other facilities, making the technique more widely available to clinical investigators. Additionally, it has been observed the development of PET cameras and availability of the instrumentation in many PET centers is increasing. Hence, it is increasingly important to develop new tracers labeled with F-18.

The PET tracers currently used in tumor diagnosis have some undisputed disadvantages: thus, FDG is preferably accumulated in cells having an elevated glucose metabolism; however, under different pathological and physiological conditions, as also in elevated glucose metabolism in the cells and tissues involved, for example infection sites or wound healing (summarized in J. Nucl. Med. Technol. (2005), 33, 145-155). Frequently, it is still difficult to ascertain whether a lesion detected via FDG-PET is really of neoplastic origin or is the result of other physiological or pathological conditions of the tissue. Overall, the diagnosis by FDG-PET in oncology has a sensitivity of 84% and a specificity of 88% (Gambhir et al., “A tabulated summary of the FDG PET literature”, J. Nucl. Med. 2001, 42, 1-93S). The imaging of brain tumors, for example, is very difficult owing to the high accumulation of FDG in healthy brain tissue.

In some cases, the F-18 labeled amino acid derivatives currently known are well suited for the detection of tumors in the brain (review): Eur. J. Nucl. Med. Mol. Imaging. 2002 May; 29(5): 681-90); however, in the case of other tumors, they are not able to compete with the imaging properties of the “Goldstandard” FDG. The metabolic accumulation and retention of the current F-18 labeled amino acids in tumor tissue is generally lower than of FDG.

Several α-L-amino acids of general formula A have been labeled with fluorine-18 so far.

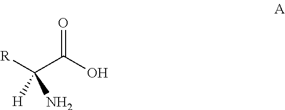

Well known are fluorine-18 labeled derivatives, whereas R is bearing an aromatic moiety, e.g.:
FDOPA
2-fluoro-L-thyrosine
OMFD
Fluoroethyl-L-tyrosine
Fluoromethyl-L-tyrosine

However, only few examples are described, wherein a F-18 labeled amino acid A is bearing a substituent R without an aromatic moiety:
S-(2-[F-18]fluoroethyl)-L-homocysteine/S-(2-[F-18]fluoroethyl)-L-methionine (1) (Bourdier et. al, Journal Labeled Compounds and Radiopharmaceuticals, 2008, 51, 369-373; Tang et. al, Nuclear Medicine and Biology, 2003, 30, 509-512.)
[F-18]fluoromethionine (2) (Neal et. al, Journal Labeled Compounds and Radiopharmaceuticals, 2005, 51, 557-368.)
[F-18]fluoroalanine (3) (Yang et al., Journal of Drug Targeting, 1993, 1, 259-267.)
O-(2-deoxy-2-[F-18]fluoro-D-glucopyranosyl)-L-serine (4), O-(2-deoxy-2-[F-18]fluoro-D-glucopyranosyl)-L-threonine (5) (Maschauer et. al, Journal Labeled Compounds and Radiopharmaceuticals, 2005, 48, 701-719.)
F-18 labeled glutamic acid derivatives (6) (WO2008052788)

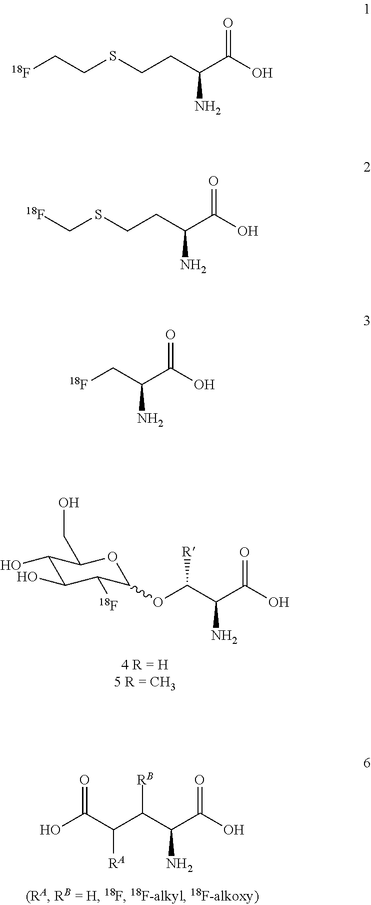

Bourdier et. al synthesized 1 by a two-step/one-pot sequence using protected S-(2-bromoethyl)-L-homocysteine or S-(2-chloroethyl)-L-homocysteine. After fluorination using [F-18]fluoride/kryptofix/potassium carbonate at 100° C., protecting groups were cleaved with 6N HCl at 100° C. After neutralization with 6N NaOH and dilution with water, the mixture was analyzed by HPLC. The authors found 1 to be unstable in aqueous solution. Repeating the synthesis method described by Tang et. al, the authors found the same instability of 1 in aqueous media.

[F-18]fluoromethionine (2) was synthesized by Neal et. al by two-step/two-pot method. After synthesis of [F-18]fluoromethyltosylate, the labeled intermediate was purified by solid-phase-extraction. After evaporation, alkylation of homocysteine afforded 14% of [F-18]fluoromethionine (2) based on the labeled intermediate [F-18]fluoromethyltosylate.

The synthesis of [F-18]fluoroalanine (3) by a two-step/two-pot sequence was described by Yang at. al. Tosylserine N-Boc methyl ester was treated with [F-18]fluoride/kryptofix/potassium carbonate in acetonitrile at 100° C. The labeled intermediate was passed through a silica gel column and the solvent was evaporated. The protecting groups were cleaved using 2N HCl at 100° C. After neutralization with 2N NaOH, the mixture was passed through a C18 cartridge and diluted with water.

O-(2-deoxy-2-[F-18]fluoro-D-glucopyranosyl)-L-serine (4) and O-(2-deoxy-2-[F-18]fluoro-D-glucopyranosyl)-L-threonine (5) were synthesized by Maschauer et al. by F-18 glycosylation of protected amino acids. Peracetylated [F-18]FDG was used a prosthetic group. The synthesis sequence started with F-18 fluorination of (1,3,4,6-tetra-O-acetyl-2-O-trifluormethanesulfonyl-(3-d-mannopyranose). The protected [F-18]FDG derivative was purified by semi-preparative HPLC. BF₃ etherate and Fmoc-protected serine or threonine was added. After heating at 80° C., the crude product mixture was purified by a second semi-preparative HPLC. Finally, protecting groups were cleaved in a third reaction vessel.

F-18 labeled glutamic acid derivatives have been disclosed in WO2008052788. High uptake of the tracers was found in several tumor cell lines.

4-(3-[F-18]fluoropropyl)-glutamic acid was prepared in a two-pot sequence: 1) [F-18]fluorination of N-Boc 4-(3bromopropyl)-glutamic acid dimethyl ester; 2) solid-phase purification on silica gel; 3) purification by preparative reversed phase HPLC; 4) solid-phase-extraction on C18 silica gel; 5) deprotection using 4N HCl; 6) Neutralization using 2N NaOH.

### PROBLEM TO BE SOLVED

However, for a routine clinical use of a [¹⁸F] labeled glutamic acid derivative, a reliable and robust manufacturing process is needed, that is compliant with compliant with Good Manufacturing Practices (GMP) requirements and provides a stable injectable solution (isotonic, neutral pH) of the radiotracer. In face of the short half-live of [¹⁸F] (110 min), the process has to provide the radiolabeled tracer in high radiochemical yield within short synthesis time (preferably less then 60 min).

[¹⁸F] labeled glutamic acid derivatives of Formula I have two stereo centers.

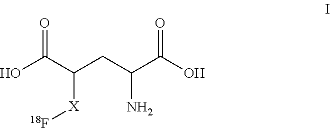

A method for manufacturing of such compounds has to assure, that the reaction conditions of the method don't lead to a significant degree of epimerization at one or both stereocenters.

The problem to be solved by the present invention was to provide a robust and reliable one-pot process for the manufacturing of an injectable formulation of [¹⁸F] labeled glutamic acid derivatives with isomeric purity of greater than 90%.

Remote controlled synthesizers for [¹⁸F] labeling should be adaptable to this process to allow a GMP compliant manufacturing of the ratio tracer.

### SUMMARY OF THE INVENTION

The present invention provides a method for production of radiolabeled compound of Formula I and suitable salts of an inorganic or organic acid thereof, hydrates, complexes, esters, amides, solvates and prodrugs thereof and optionally a pharmaceutically acceptable carrier, diluent, adjuvant or excipient.
The method comprises the steps of:
   Radiofluorination of compound of Formula II to obtain compound of Formula III,
   Cleavage of the protecting groups of compound of Formula III to obtain compound of Formula I,
   Purification and formulation of compound of Formula I by solid-phase extraction to obtain an injectable solution of compound of Formula I
wherein

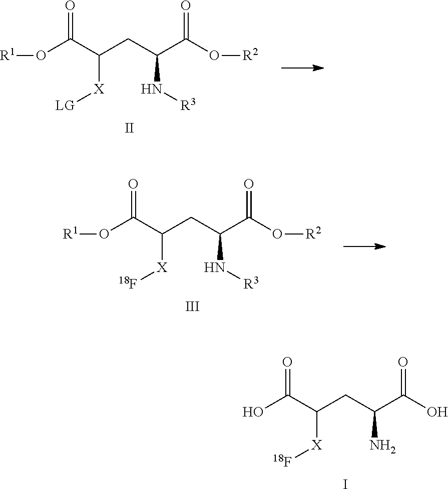

The present invention provides compounds of Formula IIa, IIb, IIc or IId which are precursors (starting material) suitable for the manufacturing process to obtain compounds of Formula Ia, Ib, Ic or Id.
The present invention also provides compounds of Formula I with isomeric purity of greater than 90%.
The present invention also provides compositions comprising a radiolabeled compound of Formula I, Ia, Ib, Ic or Id or suitable salts of an inorganic or organic acid thereof, hydrates, complexes, esters, amides, solvates and prodrugs thereof and optionally a pharmaceutically acceptable carrier, diluent, adjuvant or excipient.
The present invention also provides a kit for preparing a radiopharmaceutical preparation by the herein described process, said kit comprising a sealed vial containing a predetermined quantity of the compound of Formula II, IIa, IIb, IIc or IId.

### FIGURES

FIG. 1 Scheme of the “Eckert&Ziegler modular lab” (Adopted from Eckert&Ziegler Modular Lab software).

FIG. 2 Radio-TLC (silica, n-butanol/acetic acid/water/ethanol 12/3/5/1.5).

FIG. 3 Radio-TLC (silica, n-butanol/acetic acid/water/ethanol 12/3/5/1.5).

FIG. 4 Radio-TLC (silica, n-butanol/acetic acid/water/ethanol 12/3/5/1.5).

FIG. 5 Radio-TLC (silica, n-butanol/acetic acid/water/ethanol 12/3/5/1.5).

FIG. 6 Scheme of the “GE tracerlab FX” (Adopted from GE Tracerlab software).

Vial 14 was directly connected with the mixing vessel (the HPLC part of the module was not used).

FIG. 7 Scheme of the “GE tracerlab FX” (Adopted from GE Tracerlab software).

FIG. 8 Derivatization HPLC (Chromolith Speed ROD, 50*4.6 mm, 5 μm, Merck; 0-95% acetonitrile in 10 mM phosphate buffer (pH 7.4); 2 mL/min); Example 8a, Table 1, entry 5; top: Radioactivity signal of (2S,4S)-4-(3-[¹⁸]Fluoropropyl)-glutamic acid; bottom: co-elution, UV detection (340 nm) of ¹⁹F reference (2S,4S)-4-(3-Fluoropropyl)-glutamic acid

FIG. 9 Derivatization HPLC (Luna5μ C18(2), 250*4.6 mm, 5μ, Phenomenex; 12% acetonitrile in 10 mM phosphate buffer pH 7.4; 1.2 mL/min); Radioactivity signal; 1a: (2S,4S)-4-(3-[¹⁸F]Fluoropropyl)-glutamic acid; 1b: (2S,4R)-4-(3-[¹⁸F]Fluoropropyl)-glutamic acid

FIG. 10 Determination of identity, radiochemical purity and ratio of diastereoisomers by HPLC (Hypercarb column [100*4.6 mm, 7μ, Thermo Scientific] with 2% acetonitrile in water+0.1% TFA); A) (2S,4S)-4-(3-[¹⁸F]Fluoropropyl)-glutamic acid (1a) detected by radioactivity detector; B) reference (2S,4S)-4-(3-[¹⁹F]Fluoropropyl)-glutamic acid (1a) detected by Corona detector; C) reference (2S,4R)-4-(3-[¹⁹F]Fluoropropyl)-glutamic acid detected by Corona detector (1b); D) blank run of buffer, detection by Corona

FIG. 11 Determination of radiochemical purity and ratio of diastereoisomers by HPLC (Hypercarb column [100*4.6 mm, 7μ, Thermo Scientific] with 2% acetonitrile in water+0.1% TFA, radioactivity detector). Results from Example 10b). a) Table 2, entry 6: ratio (2S,4S)-4-(3-[18]-Fluoropropyl)-glutamic acid (1a)/(2S,4R)-4-(3-[18F]Fluoropropyl)-glutamic acid (1b)>98/2; b) Table 2, entry 10: ratio 1a/1b=95/5; c) Table 2, entry 13: ratio 1a/1b=92/8; d) Table 2, entry 16: ratio 1a/1b=82/18; e) Table 2, entry 11: ratio 1a/1b=60/40; f) Table 2, entry 17: ratio 1a/1b=50/50.

FIG. 12 Derivatization HPLC (Chromolith Speed ROD, 50*4.6 mm, 5 μm, Merck; 0-95% acetonitrile in 10 mM phosphate buffer (pH 7.4); 2 mL/min); Example 14, Radioactivity signal of (2S,4S)-4-(6-[¹⁸F]Fluorohexyl)-glutamic acid.

### DESCRIPTION OF THE INVENTION

In a first aspect the present invention is directed to a method for producing compound of Formula I

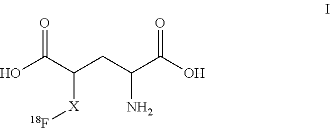

comprising the steps of:
Step 1: Synthesizing radiolabeled compound of Formula III by reacting compound of Formula II with a F-18 fluorinating agent,

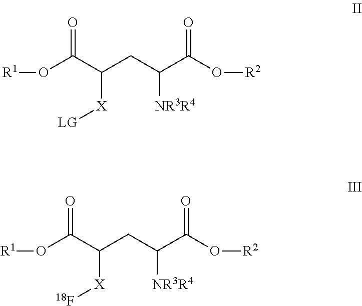

Step 2: Cleavage of the protecting groups of compound of Formula III to obtain compound of Formula I,
Step 3: Purification and formulation of compound of Formula I
   wherein:
X is selected from the group comprising
a) bond,
b) branched or non-branched (C2-C10)alkyl,
c) branched or non-branched (C2-C10)alkoxy,
d) branched or non-branched (C3-C10)alkenyl,
e) branched or non-branched (C3-C10)alkynyl,
f) [(CH₂)ₙ—O]ₘ—(CH₂)ₒ, and
g) O—[(CH₂)ₙ—O]ₘ—(CH₂)ₒ;
n=2-6, preferably n=2 or 3;
m=1-3, preferably m=1 or 2;
o=2-6, preferably o=2 or 3;

R¹ and R² are carboxyl-protecting groups and wherein carboxyl-protecting group is independently from each other selected from
a) branched or non-branched (C₁-C₆)alkyl,
b) benzyl, and
c) allyl;

R³ and R⁴ are independently from each other selected from the group comprising:
a) hydrogen,
b) an amine-protecting group or
c) the group NR³R⁴ is a 1,3-dioxo-1,3-dihydro-2H-isoindol-2-yl (phthalimido) or an azido group.

Formulas I, II and III encompass single isomers, tautomers, diastereomers, enantiomers, mixtures thereof and suitable salts thereof.

In a preferred embodiment, X is selected from the group comprising
a) a bond,
b) branched or non-branched (C3-C8)alkyl,
c) branched or non-branched (C2-C8)alkoxy,
d) branched or non-branched (C3-C8)alkenyl,
e) branched or non-branched (C3-C8)alkynyl,
f) [(CH₂)ₙ—O]ₘ—(CH₂)ₒ, and
g) O—[(CH₂)ₙ—O]ₘ—(CH₂)ₒ.

In a more preferred embodiment, X is branched or non-branched (C3-C8)alkyl and even more preferably (C3-C6)alkyl.

In a preferred embodiment, carboxyl-protecting group is Methyl, Ethyl, Propyl, Butyl, t-Butyl, or allyl. In a more preferred embodiment, carboxyl-protecting group is
a) methyl,
b) ethyl,
c) tert-butyl or
d) allyl.

Preferably, R¹ and R² are independently from each other methyl, ethyl or tert-butyl.

Amine-protecting group is Carbobenzyloxy (Cbz), p-Methoxybenzyl carbonyl (Moz or MeOZ), tert-Butyloxycarbonyl (BOC), 9-Fluorenylmethyloxycarbonyl (FMOC), Benzyl (Bn), p-Methoxybenzyl (PMB), 3,4-Dimethoxybenzyl (DMPM), Triphenylmethyl, p-methoxyphenyl (PMP), Trityl, Methoxytrityl, 1,3-dioxo-1,3-dihydro-2H-isoindol-2-yl (phthalimido) or an azido group.

In a preferred embodiment, R⁴ is hydrogen and R³ is selected from the group comprising:
a) tert-Butyloxycarbonyl (BOC),
b) Trityl and
c) Methoxytrityl.

LG is a leaving group.

In a preferred embodiment, LG is selected from the group comprising:
a) Halogen and
b) sulfonate.

Halogen is chloro, bromo or iodo. Preferably, Halogen is bromo or chloro.

Sulfonate is Methylsulfonyloxy, Trifluoromethylsulfonyloxy, (4-Nitrophenyl)sulphonyloxy, Nonafluorobutylsulfonyloxy or (4-Methylphenyl)sulfonyloxy. Preferably, Sulfonate is (4-Nitrophenyl)sulphonyloxy, Nonafluorobutylsulfonyloxy or (4-Methylphenyl)sulfonyloxy.

Compounds obtained by invention method are selected from but not limited to

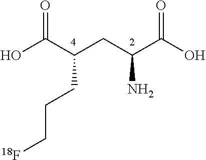 [SMILES: N[C@@H](C[C@H](CCC[18F])C(=O)O)C(=O)O]

(2S,4S)-4-{3-[F-18]fluoropropyl}-glutamic acid, and

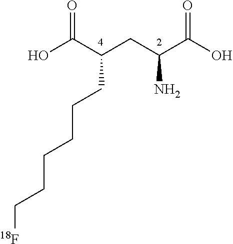 [SMILES: N[C@@H](C[C@H](CCCCCC[18F])C(=O)O)C(=O)O]

(2S,4S)-4-{3-[F-18]fluorohexyl}-glutamic acid.

Step 1 comprises a straight forward fluoro labeling reaction from compounds of Formula II for obtaining compound of formula III.

The radiolabeling method for obtaining compound of formula III comprises the step of reacting a compound of formula II with a F-18 fluorinating agent comprising a [F-18]fluoride derivative for obtaining a compound of formula III. In a preferred embodiment, the [F-18]fluoride derivative is 4,7,13,16,21,24-Hexaoxa-1,10-diazabicyclo[8.8.8]-hexacosane K[F-18]F (crownether salt Kryptofix K[F-18]F), K[F-18]F, H[F-18]F, KH[F-18]F₂, Cs[F-18]F, Na[F-18]F or tetraalkylammonium salt of [F-18]F (e.g. [F-18]tetrabutylammonium fluoride). More preferably, the fluorination agent is K[F-18]F, H[F-18]F, [F-18]tetrabutylammonium fluoride, Cs[F-18]F or KH[F-18]F₂, most preferably K[F-18], Cs[F-18]F or [F-18]tetrabutylammonium fluoride.

The radiofluorination reactions are carried out in acetonitrile, dimethylsulfoxide or dimethylformamide or a mixture thereof. But also other solvents can be used which are well known to someone skilled in the art. Water and/or alcohols can be involved in such a reaction as co-solvent. The radiofluorination reactions are conducted for less than 60 minutes. Preferred reaction times are less than 30 minutes. Further preferred reaction times are less than 15 min. This and other conditions for such radiofluorination are known to experts (Coenen, Fluorine-18 Labeling Methods Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50).

Step 2 comprises the deprotection of compound of formula III to obtain compound of formula I (cleavage). Reaction conditions are known or obvious to someone skilled in the art, which are chosen from but not limited to those described in the textbook Greene and Wuts, Protecting groups in Organic Synthesis, third edition, page 494-653, included herewith by reference. Preferred reaction conditions are addition of an acid and stirring at 0° C.-180° C.; addition of an base and heating at 0° C.-180° C.; or a combination thereof.

Preferably the step 1 and step 2 are performed in the same reaction vessel.

Step 3 comprises the purification and formulation of compound of formula I by solid-phase-extraction. Preferably solid-phase-extraction cartridges or column can be used. Suitable solid phases for trapping of compound of formula I and elution of compound of formula I from the solid phase by aqueous buffers are chosen from but not limited to cation exchange resins (e.g. Waters MCX), Oasis HLB, Hydrophilic interaction liquid chromatography (HILIC) phases (e.g. Sequant Zic-Hilic). Additionally, compound of formula I can purified by passing through solid phases chosen from but not limited to silica gel, RP silica gel, (C1-C18) silica gel, alumina, polystyrene-divinylbenzene copolymer (HR-P), hypercarb.

In a preferred embodiment, the method is carried out by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated synthesis. More preferably, the process is carried out by use of an one-pot module.

Formulas I, II and III encompass suitable salts of an inorganic or organic acid thereof, hydrates, complexes, solvates and prodrugs thereof and optionally a pharmaceutically acceptable carrier, diluent, adjuvant or excipient.

In a second aspect the present invention is directed to a fully automated and/or remote controlled method for production of compound of Formula I. In a preferred embodiment this method is a fully automated process, that provides a formulation of Formula I for the use of administration (injection) into human.

Compound of formula I is disclosed above in the first aspect.

In a third aspect the present invention is directed to a method for obtaining compound of Formula I with isomeric purity of greater than 90%, more preferably greater than 95%.

Compound of formula I is disclosed above in the first aspect.

In a fourth aspect the present invention is directed to compounds of Formula IIa, IIb, IIe or IId independently from each other

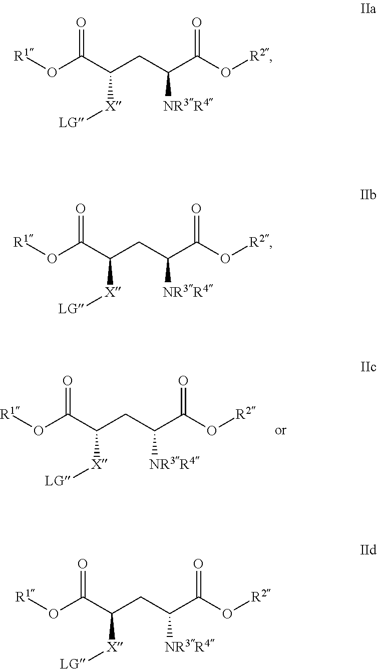

wherein:

X″ is selected from the group comprising
a) branched or non-branched (C2-C10) alkyl,
b) branched or non-branched (C2-C10) alkoxy,
c) branched or non-branched (C3-C10) alkenyl with the proviso that LG is not attached to a sp² hybridized carbon atom,
d), branched or non-branched (C3-C10) alkynyl, with the proviso that LG is not attached to a sp hybridized carbon atom,
e) [(CH₂)_{n″}—-O]_{m″}—(CH₂)_{o″}—, and
f) O—[(CH₂)_{n″}—O]_{m″}—(CH₂)_{o″};
   wherein
n″=2-6, preferably n″=2 or 3,
m″=1-3, preferably m″=1 or 2,
o″=2-6, preferably o″=2 or 3,

R^{1″} and R^{2″} are carboxyl-protecting groups and wherein carboxyl-protecting group is independently from each other selected from
a) branched or non-branched (C₁-C₆)alkyl,
b) benzyl, and
c) allyl;

R^{3″} and R^{4″} are independently from each other selected from the group comprising:
a) hydrogen,
b) an amine-protecting group or
c) the group Nee is a 1,3-dioxo-1,3-dihydro-2H-isoindol-2-yl (phthalimido) or an azido group.

Formula IIa, IIb, IIc and IId encompass single isomers, tautomers, and suitable salts of an inorganic or organic acid thereof, hydrates, complexes, solvates and prodrugs thereof and optionally a pharmaceutically acceptable carrier, diluent, adjuvant or excipient.

In a preferred embodiment, X″ is selected from the group comprising
a) branched or non-branched (C3-C6)alkyl,
b) branched or non-branched (C7-C10)alkyl,
c) branched or non-branched (C2-C8)alkoxy,
d) branched or non-branched (C3-C8)alkenyl, with the proviso that LG is not attached to a sp² hybridized carbon atom,
e), branched or non-branched (C3-C8) alkynyl, with the proviso that LG is not attached to a sp hybridized carbon atom,
f) [(CH₂)_{n″}—O]_{m″}—(CH₂)_{o″}, and
g) O—[(CH₂)_{n″}—O]_{m″}—(CH₂)_{o″}.

In a more preferred embodiment, X″ is branched or non-branched (C3-C8)alkyl, preferably (C3-C6)alkyl.

In an even more preferred embodiment, X″ is propyl.

In a preferred embodiment, carboxyl-protecting group is Methyl, Ethyl, Propyl, Butyl, t-Butyl or Allyl.

In a more preferred embodiment, carboxyl-protecting group is
a) methyl,
b) ethyl,
c) Pert-butyl or
d) allyl.

Preferably, R^{1″} and R^{2″} are independently from each other methyl, ethyl or tert-butyl.

Amine-protecting group is Carbobenzyloxy (Cbz), p-Methoxybenzyl carbonyl (Moz or MeOZ), tert-Butyloxycarbonyl (BOC), 9-Fluorenylmethyloxycarbonyl (FMOC), Benzyl (Bn), p-Methoxybenzyl (PMB), 3,4-Dimethoxybenzyl (DMPM), Triphenylmethyl, p-methoxyphenyl (PMP), Trityl, Methoxytrityl, 1,3-dioxo-1,3-dihydro-2H-isoindol-2-yl (phthalimido) or an azido group.

In a preferred embodiment, R^{4″} is hydrogen and R^{3″} is selected from the group comprising:
a) tert-Butyloxycarbonyl (BOC),
b) Trityl and
c) Methoxytrityl.

LG″ is a leaving group.

In a preferred embodiment, LG″ is selected from the group comprising:
a) Halogen and
b) sulfonate.

Halogen is chloro, bromo or iodo. Preferably, Halogen is bromo or chloro. Sulfonate is Methylsulfonyloxy, Trifluoromethylsulfonyloxy, (4-Nitrophenyl)sulphonyloxy, Nonafluorobutylsulfonyloxy or (4-Methylphenyl)sulfonyloxy. Preferably, Sulfonate is (4-Nitrophenyl)sulphonyloxy, Nonafluorobutylsulfonyloxy or (4-Methylphenyl)sulfonyloxy.

Preferably, the compounds of fourth aspect are directed to compounds of formula IIa.

Preferably, the compounds of fourth aspect are directed to compounds of formula IIb.

Preferably, the compounds of fourth aspect are directed to compounds of formula IIc.

Preferably, the compounds of fourth aspect are directed to compounds of formula IId.

Invention compounds IIa are selected from but not limited to

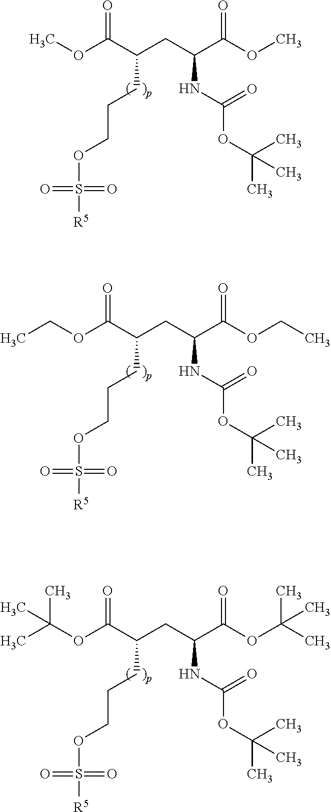

wherein

p=1-4 and

R⁵ is selected from the group comprising
a) optionally substituted branched or non-branched alkyl, and
b) optionally substituted aryl or heteroaryl.

In a preferred embodiment, R⁵ is selected from the group comprising
a) methyl,
b) trifluoromethyl,
c) p-methylphenyl,
d) nitrophenyl,
e) bromophenyl and
f) nonafluorobutyl.

A more preferred compound of Formula IIa is:

Di-tert-butyl (2S,4S)—N-(tert-butoxycarbonyl)-4-[3-(mesyloxy])propy]-glutamate

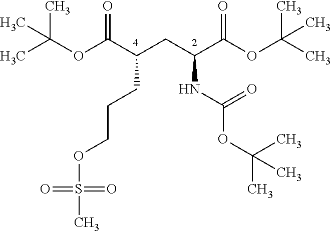 [SMILES: CC(C)(C)OC(=O)N[C@@H](C[C@H](CCCOS(C)(=O)=O)C(=O)OC(C)(C)C)C(=O)OC(C)(C)C]

Another more preferred compound of Formula IIa is:

Di-tert-butyl (2S,4S)—N-(tert-butoxycarbonyl)-4-[3-(tosyloxy)propy]-glutamate

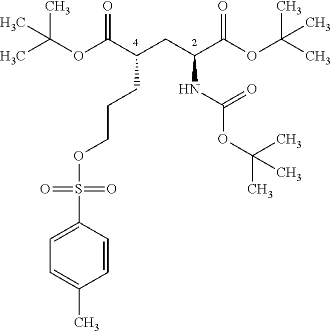 [SMILES: Cc1ccc(S(=O)(=O)OCCC[C@@H](C[C@H](NC(=O)OC(C)(C)C)C(=O)OC(C)(C)C)C(=O)OC(C)(C)C)cc1]

Another more preferred compound of Formula IIa is:

Di-tert-butyl (2S,4S)—N-(tert-butoxycarbonyl)-4-(3-{[(4-nitrophenyl)-sulfonyl]oxy}propyl)-glutamate

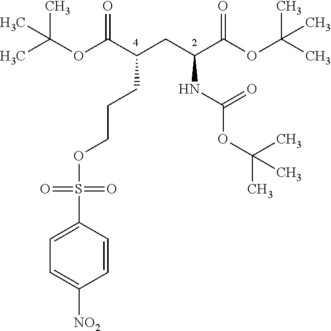 [SMILES: CC(C)(C)OC(=O)N[C@@H](C[C@H](CCCOS(=O)(=O)c1ccc([N+](=O)[O-])cc1)C(=O)OC(C)(C)C)C(=O)OC(C)(C)C]

Another more preferred compound of Formula IIa is:

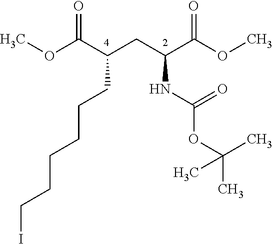 [SMILES: COC(=O)[C@@H](CCCCCCI)C[C@H](NC(=O)OC(C)(C)C)C(=O)OC]

Invention compounds IIb are selected from but not limited to

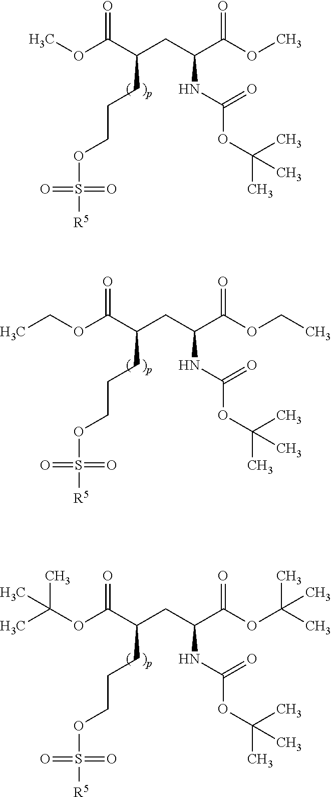

wherein

p=1-4 and

R⁵ is selected from the group comprising
a) optionally substituted branched or non-branched alkyl, and
b) optionally substituted aryl or heteroaryl.

In a preferred embodiment, R⁵ is selected from the group comprising
a) methyl,
b) trifluoromethyl,
c) p-methylphenyl,
d) nitrophenyl,
e) bromophenyl and
f) nonafluorobutyl.

A more preferred compound of Formula IIb is:

Di-tert-butyl (2S,4R)—N-(tert-butoxycarbonyl)-4-[3-(mesyloxyl])propy]-glutamate

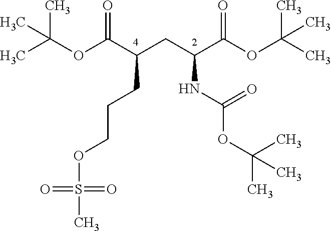 [SMILES: CC(C)(C)OC(=O)N[C@@H](C[C@@H](CCCOS(C)(=O)=O)C(=O)OC(C)(C)C)C(=O)OC(C)(C)C]

Another more preferred compound of Formula IIb is:

Di-tert-butyl (2S,4R)—N-(tert-butoxycarbonyl)-4-[3-(tosyloxy)propy]-glutamate

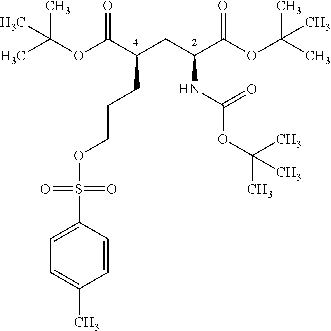 [SMILES: Cc1ccc(S(=O)(=O)OCCC[C@H](C[C@H](NC(=O)OC(C)(C)C)C(=O)OC(C)(C)C)C(=O)OC(C)(C)C)cc1]

Another more preferred compound of Formula IIb is:

Di-tert-butyl(2S,4R)—N-(tert-butoxycarbonyl)-4-(3-{[(4-nitrophenyl)-sulfonyl]oxy}propyl)-glutamate

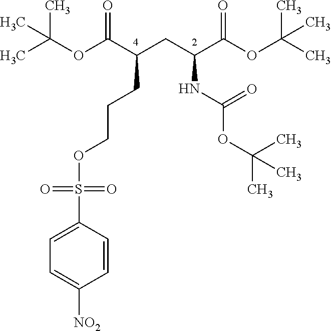 [SMILES: CC(C)(C)OC(=O)N[C@@H](C[C@@H](CCCOS(=O)(=O)c1ccc([N+](=O)[O-])cc1)C(=O)OC(C)(C)C)C(=O)OC(C)(C)C]

Invention compounds IIc are selected from but not limited to

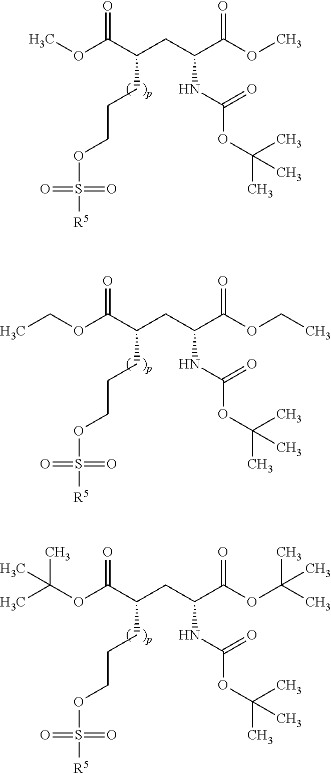

wherein

p=1-4 and

R⁵ is selected from the group comprising
a) optionally substituted branched or non-branched alkyl, and
b) optionally substituted aryl or heteroaryl.

In a preferred embodiment, R⁵ is selected from the group comprising
a) methyl,
b) trifluoromethyl,
c) p-methylphenyl,
d) nitrophenyl,
e) bromophenyl and
f) nonafluorobutyl.

A more preferred compound of Formula IIc is:

Di-tert-butyl (2R,4S)—N-(tert-butoxycarbonyl)-4-[3-(mesyloxy])propy]-glutamate

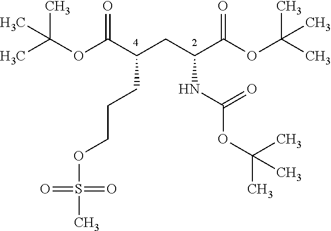 [SMILES: CC(C)(C)OC(=O)N[C@H](C[C@H](CCCOS(C)(=O)=O)C(=O)OC(C)(C)C)C(=O)OC(C)(C)C]

Another more preferred compound of Formula IIc is:

Di-tert-butyl (2R,4S)—N-(tert-butoxycarbonyl)-4-[3-(tosyloxy)propy]-glutamate

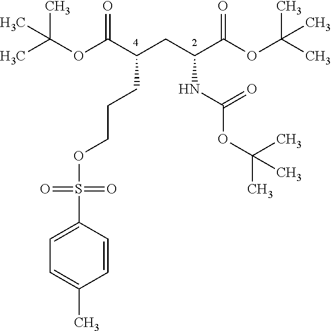 [SMILES: Cc1ccc(S(=O)(=O)OCCC[C@@H](C[C@@H](NC(=O)OC(C)(C)C)C(=O)OC(C)(C)C)C(=O)OC(C)(C)C)cc1]

Another more preferred compound of Formula IIc is:

Di-tert-butyl (2R,4S)—N-(tert-butoxycarbonyl)-4-(3-{[(4-nitrophenyl)-sulfonyl]oxy}propyl)-glutamate

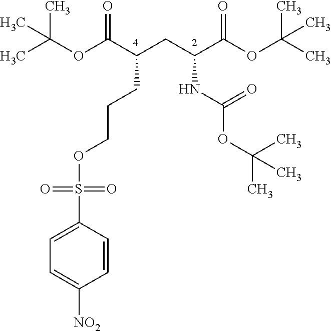 [SMILES: CC(C)(C)OC(=O)N[C@H](C[C@H](CCCOS(=O)(=O)c1ccc([N+](=O)[O-])cc1)C(=O)OC(C)(C)C)C(=O)OC(C)(C)C]

Invention compounds IId are selected from but not limited to

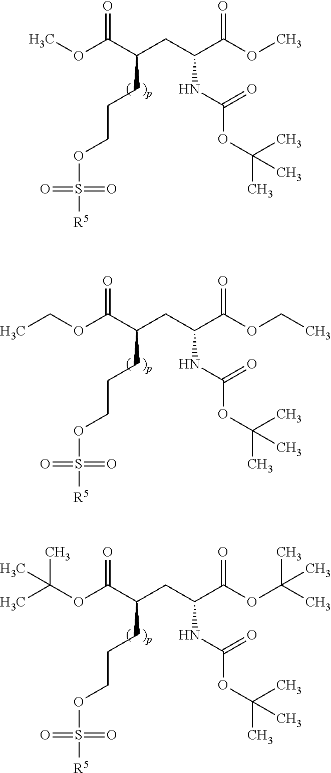

wherein

p=1-4 and

R⁵ is selected from the group comprising
a) optionally substituted branched or non-branched alkyl, and
b) optionally substituted aryl or heteroaryl.

In a preferred embodiment, R⁵ is selected from the group comprising
a) methyl,
b) trifluoromethyl,
c) p-methylphenyl,
d) nitrophenyl,
e) bromophenyl and
f) nonafluorobutyl.

A more preferred compound of Formula IId is:

Di-tert-butyl (2R,4R)—N-(tert-butoxycarbonyl)-4-[3-(mesyloxy])propy]-glutamate

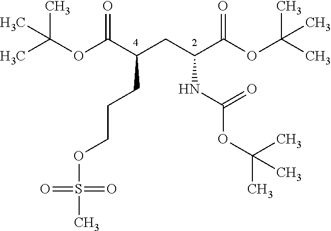 [SMILES: CC(C)(C)OC(=O)N[C@H](C[C@@H](CCCOS(C)(=O)=O)C(=O)OC(C)(C)C)C(=O)OC(C)(C)C]

Another more preferred compound of Formula IId is:

Di-tert-butyl (2R,4R)—N-(tert-butoxycarbonyl)-4-[3-(tosyloxy)propy]-glutamate

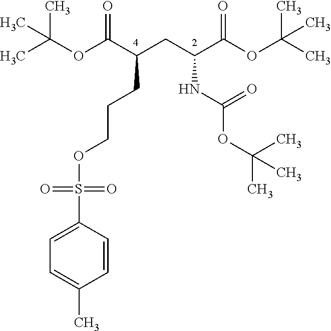 [SMILES: Cc1ccc(S(=O)(=O)OCCC[C@H](C[C@@H](NC(=O)OC(C)(C)C)C(=O)OC(C)(C)C)C(=O)OC(C)(C)C)cc1]

Another more preferred compound of Formula IId is:

Di-tert-butyl (2R,4R)—N-(tert-butoxycarbonyl)-4-(3-{[(4-nitrophenyl)-sulfonyl]oxy}propyl)-glutamate

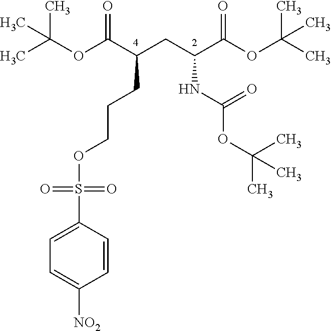 [SMILES: CC(C)(C)OC(=O)N[C@H](C[C@@H](CCCOS(=O)(=O)c1ccc([N+](=O)[O-])cc1)C(=O)OC(C)(C)C)C(=O)OC(C)(C)C]

In a fifth aspect the present invention is directed to compounds of Formula Ia, Ib, Ic or Id independently from each other

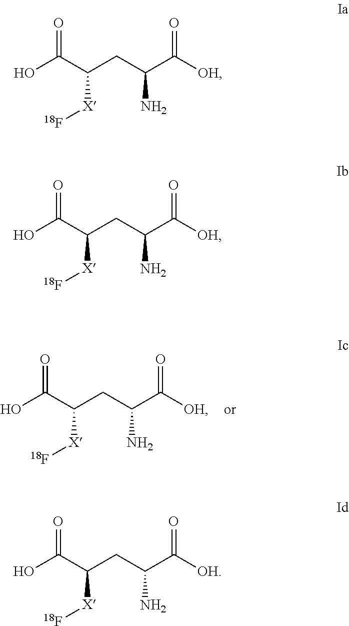

wherein:

X′ is selected from the group comprising
a) branched or non-branched (C2-C10)alkyl,
b) branched or non-branched (C2-C10)alkoxy,
c) branched or non-branched (C3-C10) alkenyl with the proviso that LG is not attached to a sp² hybridized carbon atom,
d), branched or non-branched (C3-C10) alkynyl, with the proviso that LG is not attached to a sp hybridized carbon atom,
e) [(CH₂)_{n′}—O]_{m′}—(CH₂)_{o′}, and
f) O—[(CH₂)_{n′}—O]_{m′}—(CH₂)_{o′};
n′=2-6, preferably n′=2 or 3;
m′=1-3, preferably m′=1 or 2;
o′=2-6, preferably o′=2 or 3;

Formulas Ia, Ib, Ic and Id encompass single isomers, tautomers, diastereomers, enantiomers, mixtures thereof and suitable salts of an inorganic or organic acid thereof, hydrates, complexes, solvates and prodrugs thereof and optionally a pharmaceutically acceptable carrier, diluent, adjuvant or excipient.

In a preferred embodiment, X′ is selected from the group comprising
a) branched or non-branched (C3-C6)alkyl,
b) branched or non-branched (C7-C10)alkyl,
c) branched or non-branched (C2-C8)alkoxy,
d) branched or non-branched (C3-C8)alkenyl, with the proviso that LG is not attached to a sp² hybridized carbon atom,
e), branched or non-branched (C3-C8) alkynyl, with the proviso that LG is not attached to a sp hybridized carbon atom,
f) [(CH₂)_{n′}—O]_{m′}—(CH₂)_{o′}, and
   g) O—[(CH₂)_{n′}—O]_{m′}—(CH₂)_{o′}.

In a more preferred embodiment, X′ is branched or non-branched (C3-C8)alkyl, preferably (C3-C6)alkyl.

In an even more preferred embodiment, X′ is propyl.

Preferably, the compounds of fifth aspect are directed to compound of formula Ia.

Preferably, the compounds of fifth aspect are directed to compound of formula Ib.

Preferably, the compounds of fifth aspect are directed to compound of formula Ic.

Preferably, the compounds of fifth aspect are directed to compound of formula Id.

A preferred compound of Formula Ia is:

(2S,4R)-4-(3-[¹⁸F]fluorohexyl)-glutamic acid

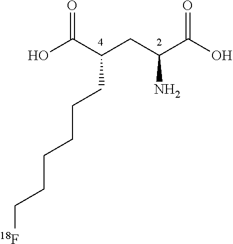 [SMILES: N[C@@H](C[C@H](CCCCCC[18F])C(=O)O)C(=O)O]

A preferred compound of Formula Ib is:

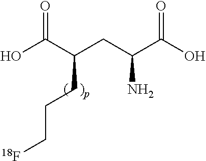

wherein

p is 1-4.

A more preferred compound of Formula Ib is:

(2S,4R)-4-(3-[¹⁸F]Fluoropropyl)-glutamic acid

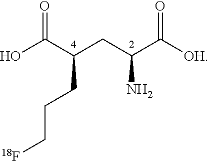 [SMILES: N[C@@H](C[C@@H](CCC[18F])C(=O)O)C(=O)O]

A preferred compound of Formula Ic is:

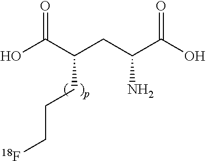

wherein

p is 1-4.

A more preferred compound of Formula Ic is:

(2R,4S)-4-(3-[¹⁸F]Fluoropropyl)-glutamic acid

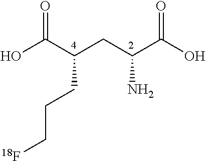 [SMILES: N[C@H](C[C@H](CCC[18F])C(=O)O)C(=O)O]

A preferred compound of Formula Id is:

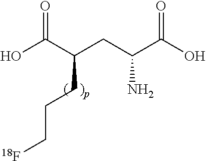

wherein

p is 1-4.

A more preferred compound of Formula Id is:

(2R,4R)-4-(3-[¹⁸F]-Fluoropropyl)-glutamic acid

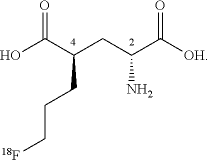 [SMILES: N[C@H](C[C@@H](CCC[18F])C(=O)O)C(=O)O]

In a sixth aspect the present invention is directed to compounds of Formula IIIa, IIIb, IIIc or IIId independently from each other

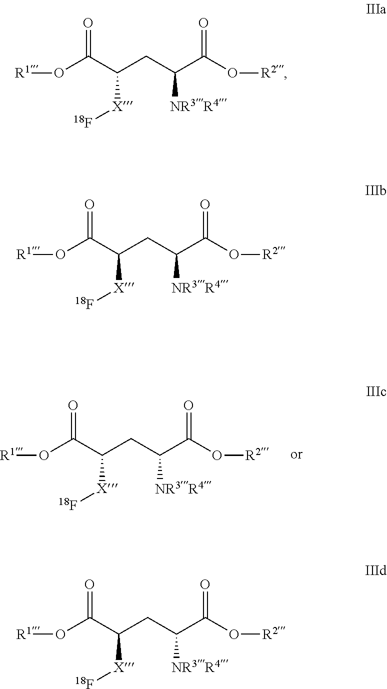

wherein:

X′″ is selected from the group comprising
a) branched or non-branched (C2-C10)alkyl,
b) branched or non-branched (C2-C10)alkoxy,
c) branched or non-branched (C3-C10) alkenyl with the proviso that ¹⁸F is not attached to a sp² hybridized carbon atom,
d), branched or non-branched (C3-C10) alkynyl, with the proviso that ¹⁸F is not attached to a sp hybridized carbon atom,
e) [(CH₂)_{n′″}—O]_{m′″}—(CH₂)_{o′″}—, and
f) O—[(CH₂)_{n′″}O]_{m′″}—(CH₂)_{o′″};
n′″=2-6, preferably n′″=2 or 3;
m′″=1-3, preferably m′″=1 or 2;
o′″=2-6, preferably o′″=2 or 3;

R^{1′″} and R^{2′″} are carboxyl-protecting groups and wherein carboxyl-protecting group is independently from each other selected from
a) branched or non-branched (C₁-C₆)alkyl,
b) benzyl, and
c) allyl;

R^{3′″} and R^{4′″} are independently from each other selected from the group comprising:
a) hydrogen,
b) an amine-protecting group or
c) the group NR^{3′″}R^{4′″} is a 1,3-dioxo-1,3-dihydro-2H-isoindol-2-yl (phthalimido) or an azido group.

Formulas IIIa, IIIb, IIIc and IIId encompass single isomers, tautomers, diastereomers, enantiomers, mixtures thereof and suitable salts of an inorganic or organic acid thereof, hydrates, complexes, solvates and prodrugs thereof and optionally a pharmaceutically acceptable carrier, diluent, adjuvant or excipient.

In a preferred embodiment, X′″ is selected from the group comprising
a) branched or non-branched (C3-C6)alkyl,
b) branched or non-branched (C7-C10)alkyl,
c) branched or non-branched (C2-C8)alkoxy,
d) branched or non-branched (C3-C8)alkenyl, with the proviso that ¹⁸F is not attached to a sp² hybridized carbon atom,
e), branched or non-branched (C3-C8) alkynyl, with the proviso that ¹⁸F is not attached to a sp hybridized carbon atom,
f) [(CH₂)_{n′″}—O]_{m′″}—(CH₂)_{o′″}, and
g) O—[(CH₂)_{n′″}—O]_{m′″}—(CH₂)_{o′″}.

In a more preferred embodiment, X′″ is

branched or non-branched (C3-C8)alkyl, preferably, (C3-C6)alkyl.

In an even more preferred embodiment, X′″ is propyl.

In a preferred embodiment, carboxyl-protecting group is Methyl, Ethyl, Propyl,

Butyl, t-Butyl, or allyl. In a more preferred embodiment, carboxyl-protecting group is
a) methyl,
b) ethyl,
c) tert-butyl or
d) allyl.

Preferably, R^{1′″} and R^{2′″} are independently from each other methyl, ethyl or tert-butyl.

Amine-protecting group is Carbobenzyloxy (Cbz), p-Methoxybenzyl carbonyl (Moz or MeOZ), tert-Butyloxycarbonyl (BOC), 9-Fluorenylmethyloxycarbonyl (FMOC), Benzyl (Bn), p-Methoxybenzyl (PMB), 3,4-Dimethoxybenzyl (DMPM), Triphenylmethyl, p-methoxyphenyl (PMP), Trityl, Methoxytrityl, 1,3-dioxo-1,3-dihydro-2H-isoindol-2-yl (phthalimido) or an azido group.

In a preferred embodiment, R^{4′″} is hydrogen and R^{3′″} is selected from the group comprising:
a) tert-Butyloxycarbonyl (BOC),
b) Trityl and
c) Methoxytrityl.

Preferably, the compounds of sixth aspect are directed to compounds of formula IIIa.

Preferably, the compounds of sixth aspect are directed to compounds of formula IIIb.

Preferably, the compounds of sixth aspect are directed to compounds of formula IIIc.

Preferably, the compounds of sixth aspect are directed to compounds of formula IIId.

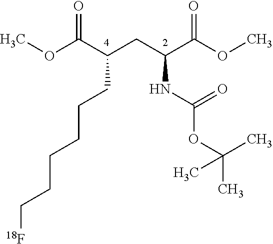 [SMILES: COC(=O)[C@@H](CCCCCC[18F])C[C@H](NC(=O)OC(C)(C)C)C(=O)OC]

In a seventh aspect the present invention is directed to a method for producing compound of Formula I wherein compound of Formula I is a compound of Formula Ia, Ib, Ic or Id comprising the steps of:
Step 1: Synthesizing radiolabeled compound of Formula III wherein compound of Formula III is a compound of Formula IIIa, IIIb, IIIc or IIId by reacting compound of Formula II wherein compound of Formula II is a compound of Formula IIa, IIb, IIc or IId with a F-18 fluorinating agent,
Step 2: Cleavage of the protecting groups of compound of Formula III wherein compound of Formula III is a compound of Formula IIIa, IIIb, IIIc or IIId to obtain compound of Formula I wherein compound of Formula I is a compound of Formula Ia, Ib, Ic or Id, and
Step 3: Purification and formulation of compound of Formula I wherein compound of Formula I is a compound of Formula Ia, Ib, Ic or Id
   wherein Step 1, Step 2, and Step 3 are described in the first aspect, compound of Formula Ia, Ib, Ic or Id, compound of Formula IIa, IIb, IIc or IId and compound of Formula IIIa, IIIb, IIIc or IIId as described in the fourth, fifth, and sixth aspects respectively.

Preferably the method is directed to compounds of formula Ia, IIa, and IIIa.

Preferably the method is directed to compounds of formula Ib, IIb, and IIIb.

Preferably the method is directed to compounds of formula Ic, IIc, and IIId.

Preferably the method is directed to compounds of formula Id, IId, and IIId.

Preferred features and embodiments disclosed above are herein enclosed.

In an eighth aspect the present invention is directed to a method for producing compounds of Formula I, Formula Ia, Formula Ib, Formula Ic, or Formula Id as described in the first, second, third, fifth and seventh aspects,

wherein the F-18 fluorination reaction described in Step 1 is carried out at 0° C.-160° C., preferably at 0° C.-140° C., more preferably at 20° C.-120° C., even more preferably at 60° C.-120° C. and even more preferably at 60° C.-100° C.

Preferably the method is directed to compounds of formula Ia.

Preferably the method is directed to compounds of formula Ib.

Preferably the method is directed to compounds of formula Ic.

Preferably the method is directed to compounds of formula Id.

More preferably, the method is automated and/or remote controlled.

In a ninth aspect the present invention is directed to a method for producing compounds of Formula I, Formula Ia, Formula Ib, Formula Ic, or Formula Id,

as described in the first, second, third, fifth and seventh aspects wherein the [¹⁸F] fluorination agent used in Step 1 is generated from a base and [¹⁸F]fluoride.

Typically, [¹⁸F]fluoride is trapped on an anion exchange resin and afterwards washed from the resin using a solution of the base into a reaction vessel. Alternatively, [¹⁸F]fluoride and the base can be mixed directly in the reaction vessel.

The base can be an inorganic or organic base.

Preferably, the base is selected from the group comprising:
a) a potassium salt,
b) a caesium salt,
c) a rubidium salt,
d) a tetraalkyl ammonium salt,
e) a phosphazene.

More preferably, the base is selected from the group comprising:
a) a potassium salt,
b) a caesium salt,
c) a tetraalkyl ammonium salt.

Even more preferably, the base is selected from the group comprising: potassium carbonate, potassium bicarbonate, tripotassium phosphate, dipotassium phosphate, monopotassium phosphate, potassium oxalate, potassium hydroxide, potassium mesylate, caesium carbonate, cesium bicarbonate, tetraalkyl ammonium hydroxide, tetraalkyl ammonium bicarbonate, tetraalkyl ammonium mesylate.

In a preferred embodiment, the ratio of the base and compound of Formula II or Formula IIa or Formula IIb or Formula IIc or Formula IId is greater than zero (>0) and equal or below 1 (≦1). More preferably, the ratio of the base and compound of Formula II or Formula IIa or Formula IIb or Formula IIc or Formula IId is greater than zero (>0) and is below 1 (<1).

Preferably the method of ninth aspect is combined with the method of eighth aspect.

In a tenth aspect the present invention is directed to a method for producing compound of Formula I, Formula Ia, Formula Ib, Formula Ic or Formula Id, as described in the first, fifth, seventh eighth and ninth aspects wherein the compound of Formula I, Formula Ia, Formula Ib, Formula Ic or Formula Id has an isomeric purity of greater than 90%, preferably greater than 95%, more preferably greater than 98%.

More preferably, the method is automated and/or remote controlled.

In an eleventh aspect the present invention is directed to a method for obtaining a formulation of compound of Formula I, Formula Ia, Formula Ib, Formula Ic or Formula Id, or mixture thereof as described in the first and fifth aspects, and invention is as well directed to a formulation of compound of Formula I, Formula Ia, Formula Ib, Formula Ic or Formula Id, or mixture thereof wherein the formulation is a radiopharmaceutical formulation suitable for the administration into mammal.

Optionally, the radiopharmaceutical formulation comprises additionally one or more physiologically acceptable vehicle or carrier and optional adjuvants and preservatives known in the art such as water and/or inorganic salts, selected from but not limited to the group comprising sodium chloride, monosodium phosphate, disodium phosphate, trisodium phosphate, any pH adjusting agent known in the art

Optionally, the radiopharmaceutical formulation comprises additionally radiostabilizers selected from but not limited to the group comprising ascorbic acid and salts thereof, gentisic acid and salts thereof.

Optionally, the radiopharmaceutical formulation comprises additionally 0-20% ethanol, preferably 0-15% ethanol, more preferably 0-10% ethanol, even more preferably less than 5% ethanol.

The method for obtaining a formulation comprising compound of Formula I, Formula Ia, Formula Ib, Formula Ic or Formula Id, or mixture thereof comprises the step of adding one or more physiologically acceptable vehicle or carrier, adjuvants or preservatives known in the art as listed above to a solution of compound of Formula I, Formula Ia, Formula Ib, Formula Ic or Formula Id, or mixture thereof wherein the formulation is suitable for the administration into mammal.

In a twelfth aspect the present invention is directed to a device for carrying out the method as described in first, second, third, seventh, eighth, ninth and tenth aspects for producing compound of Formula I, Formula Ia, Formula Ib, Formula Ic or Formula Id as described in the first and fifth aspects and radiopharmaceutical formulation as described in eleventh aspect wherein the method is an automated and/or remote controlled method or process and the invention is as well directed to the use of such device for obtaining invention compounds.

Preferably, the method or process is an automated (optionally fully automated) and/or remote controlled method or process.

The device of the present invention is a radiopharmaceutical synthesizer suitable for radiofluorinations. The device is a non-cassette type or a cassette-type synthesizers. Non-cassette type synthesizers are: GE Tracerlab FX, Eckert&Ziegler modular lab, Siemens Explora, Raytest SynChrom, Scintomics Hotbox. Cassette type synthesizers are: GE Tracerlab MX, GE Fastlab, IBA Synthera. The present invention is not limited to the mentioned synthesizers. The device of the present invention is a non-cassette type or a cassette-type synthesizers suitable for radiofluorinations charactherised in that the device is carrying out the method as described in first, second, third, seventh, eighth, ninth and tenth aspects for producing compound of Formula I, Formula Ia, Formula Ib, Formula Ic or Formula Id as described in the first and fifth aspects and radiopharmaceutical formulation as described in eleventh aspect wherein the method is an automated and/or remote controlled method or process.

In a thirteenth aspect the present invention is directed to a Kit for producing of compound of Formula I, Formula Ia, Formula Ib, Formula Ic or Formula Id, as described in the first and fifth aspects and at least one sealed vial containing a compound of formula II, Formula IIa, Formula IIb, Formula IIc or Formula IId thereof.

Preferably, the Kit comprises predefined quantity of compound of Formula II, Formula IIa, Formula IIb, Formula IIc or Formula IId as described in the first and fifth aspects and one or more solid-phase extraction cartridges/columns for the purification of compound of Formula I, Ia, Ib, Ic or Id.

Preferably, the Kit comprises physiologically acceptable vehicle or carrier and optional adjuvants and preservatives, reagents suitable to perform the herein disclosed reactions and/or [¹⁸F] labelling reagents. Furthermore, the kit may contain instructions for its use.

Optionally, the Kit comprises additionally one or more solid-phase-extraction cartridges or columns for purification of compound of Formula I, Formula Ia, Formula Ib, Formula Ic or Formula Id as described in the first and fourth aspects, preferably, at least one solid-phase-extraction cartridge or column based on cation-exchange material.

### DEFINITIONS

If chiral centers or other forms of isomeric centers are present in a compound according to the present invention, all forms of such stereoisomers, including enantiomers and diastereoisomers, are intended to be covered herein. Compounds containing chiral centers may be used as racemic mixture or as an enantiomerically enriched mixture or as a diastereomeric mixture or as a diastereomerically enriched mixture, or these isomeric mixtures may be separated using well-known techniques, and an individual stereoisomer maybe used alone. In cases in which compounds have carbon-carbon double bonds, both the (Z)-isomers and (E)-isomers as well as mixtures thereof are within the scope of this invention. In cases wherein compounds may exist in tautomeric forms, such as keto-enol tautomers, each tautomeric form is contemplated as being included.

In the context of the present invention, preferred salts are pharmaceutically suitable salts of the compounds according to the invention. The invention also comprises salts which for their part are not suitable for pharmaceutical applications, but which can be used, for example, for isolating or purifying the compounds according to the invention.

Pharmaceutically suitable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Pharmaceutically suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N methylmorpholine, arginine, lysine, ethylenediamine and N methylpiperidine.

The term halogen or halo refers to Cl, Br, F or I.

The term “C2-C10 alkyl”, used herein on its own or as part of another group, refers to saturated carbon chains which may be straight-chain or branched, in particular to ethyl, n-propyl, isopropyl, n-butyl, isobutyl, tert-butyl, n-pentyl, 2,2 dimethylpropyl, 2 methylbutyl, 3 methylbutyl, n-hexyl, n-heptyl, n-octyl, n nonyl or n-decyl groups. Preferably, C2-C10 alkyl is C2-C6 alkyl or C7-C10 alkyl. C2-C6 alkyl is preferably C3-C6 alkyl. C7-C10 alkyl is preferably C7-C8 alkyl or C9-C10 alkyl. Preferably, C2-C6 alkyl is C3-C4 alkyl, C3 alkyl or C4 alkyl.

The term “C3-C6 cycloalkyl” used herein on its own or as part of another group, refers to a cyclopropyl, cyclobutyl, cyclopentyl or cyclohexyl group.

The term “C2-C10-alkoxy” used herein on its own or as part of another group, refers to an O-alkyl chain, in particular to ethoxy, n-propoxy, n-butoxy, n-pentyloxy, n-hexyloxy, n-heptyloxy, n-octyloxy, n-nonyloxy or n-decyloxy group. Preferably, C2-C10 alkoxyl is C2-C6 alkoxyl or C7-C10 alkoxyl. C2-C6 alkoxyl is preferably C3-C6 alkoxyl. C7-C10 alkoxyl is preferably C7-C8 alkoxyl or C9-C10 alkoxyl.

The term “aryl” as employed herein by itself or as part of another group refers to phenyl or naphthyl groups, which themselves can be substituted with one, two or three substituents independently and individually selected from but not limited to the group comprising halogen, nitro, formyl, acetyl, alkoxycarbonyl, cyano, nitrile, trifluoromethyl, (C1-C3)alkylsulfonyl or (C1-C3)alkyl.

The term “heteroaryl” as employed herein by itself or as part of another group refers to monocyclic or bicyclic heteroaromatic groups containing from 5 to 10 ring atoms, wherein 1 or 2 atoms of the ring portion are independently selected from N, O or S, e.g. thienyl, furanyl, pyrrolyl, oxazolyl, thiazolyl, imidazolyl, pyrazolyl, isoxazolyl, isothiazolyl, benzofuranyl, benzothienyl, benzothiazolyl, benzoxazolyl, benzimidazolyl, indazolyl, indolyl, pyridyl, pyridazinyl, pyrimidinyl, pyrazinyl, quinolyl, isoquinolyl etc.

As outlined above such “heteroaryl” may additionally be substituted by one or two substituents independently and individually selected from but not limited to the group comprising halogen, nitro, formyl, acetyl, alkoxycarbonyl, cyano, nitrile, trifluoromethyl, (C1-C3)alkylsulfonyl or (C1-C3)alkyl.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference. The amine-protecting group is preferably Carbobenzyloxy (Cbz), p-Methoxybenzyl carbonyl (Moz or MeOZ), tert-Butyloxycarbonyl (BOC), 9-Fluorenylmethyloxycarbonyl (FMOC), Benzyl (Bn), p-Methoxybenzyl (PMB), 3,4-Dimethoxybenzyl (DMPM), p-methoxyphenyl (PMP) or the protected amino group is a 1,3-dioxo-1,3-dihydro-2H-isoindol-2-yl (phthalimido) or an azido group.

Carboxyl-protecting group is Methyl, Ethyl, Propyl, Butyl, t-Butyl, Allyl, Benzyl, 4-Methoxybenzyl or 4-Methoxyphenyl.

The term “leaving group” as employed herein by itself or as part of another group is known or obvious to someone skilled in the art, and means that an atom or group of atoms is detachable from a chemical substance by a nucleophilic agent. Examples are given e.g. in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O— nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33).

“Alkynyl” means a linear hydrocarbon radical of two to six carbon atoms or a branched hydrocarbon radical of three to 6 carbon atoms which radical contains at least one triple bond, e.g., ethynyl, propynyl, butynyl, pentyn-2-yl and the like.

“Alkenyl” means a means a linear hydrocarbon radical of two to six carbon atoms or a branched hydrocarbon radical of three to 6 carbon atoms which radical contains at least one double bond, e.g., ethenyl, propenyl, I-but-3-enyl, and 1-pent-3-enyl, and the like.

The term “purification” as employed herein has the objective to eliminate the excess of side product such as ¹⁸F-Fluoride and to concentrate and trap the reaction product. Purification is carried out by by any method known to those in the art, suitable for radiotracer e.g. solid-phase-extraction cartridges or column.

The wording “automated and/or remote controlled device” refers to a device that is suitable for carrying out the radiosynthesis of a radiolabeled compound and maybe fully automated. The device comprises a reactor system, valves modules and a controller adapted to control the operation of said network.

The term “Good manufacturing practice” or “GMP” is part of a quality system covering the manufacture and testing of active pharmaceutical ingredients, diagnostics, foods, pharmaceutical products, and medical devices. GMPs are guidelines that outline the aspects of production and testing that can impact the quality of a product e.g. manufacturing processes are clearly defined and controlled. All critical processes are validated to ensure consistency and compliance with specifications.

Unless otherwise specified, when referring to the compounds of formula the present invention per se as well as to any pharmaceutical composition thereof the present invention includes all of the hydrates, salts, and complexes.

### General Synthesis of Precursors for Radiolabeling

Precursors for alkyl-F-18 compounds of general formula I are e.g. tosylates, brosylates, nosylates, mesylates, triflates, nonaflates etc. (formula II) which can be synthesized from the respective hydroxy compounds according to methods known in the art (J. March, Advanced Organic Chemistry, 4th ed. 1992, John Wiley & Sons, pp 352ff). More specifically, a hydroxy group being attached to a sp3 hybridized carbon atom can be converted to a leaving group by an activating agent like thionyl chloride (e.g. Organic and Biomolecular Chemistry; 4; 22; (2006); 4101-4112), phosphorus pentachloride (e.g. Bioorganic and Medicinal Chemistry; 16; 6; (2008); 3309-3320), methanesulfonyl chloride (e.g. Organic and Biomolecular Chemistry; English; 4; 24; (2006); 4514-4525), carbon tetrachloride/triphenylphosphine (Tetrahedron: Asymmetry; English; 19; 5; 2008; 577-583), hydrogen chloride (e.g. Russian Chemical Bulletin; English; 56; 6; 2007; 1119-1124), N-chloro-succinimide/dimethylsulfide (e.g. Bioscience, Biotechnology, and Biochemistry 72; 3; (2008); 851-855), hydrogen bromide (e.g. Journal of Labeled Compounds and Radiopharmaceuticals; 51; 1; (2008); 12-18), phosphorus tribromide (Journal of the American Chemical Society; 130; 12; (2008); 3726-3727), carbon tetrabromide/triphenylphosphine (e.g. Journal of the American Chemical Society; 130; 12; (2008); 4153-4157), N-bromo-succimide/SMe2 (e.g. Chemical Communications (Cambridge, United Kingdom); 1; (2008); 120-122), bromine/triphenylphosphine (e.g. Journal of the American Chemical Society; 130; 12; (2008); 4153-4157), N-bromo-succimide/SMe2 (e.g. Chemical Communications (Cambridge, United Kingdom); 1; (2008); 120-122), Br2/PPh3 (e.g. European Journal of Organic Chemistry; 9; (2007); 1510-1516), mesylchloride, tosylchloride, trifluormethylsulfonylchloride, nona-fluorobutylsulfonylchloride, (4-bromo-phenyl)sulfonylchloride, (4-nitro-phenyl)sulfonylchloride, (2-nitro-phenyl)sulfonylchloride, (4-isopropyl-phenyl)sulfonylchloride, (2,4,6-tri-isopropyl-phenyl)sulfonylchloride, (2,4,6-trimethyl-phenyl)sulfonylchloride, (4-tertbutyl-phenyl)sulfonylchloride, (4-methoxy-phenyl)sulfonylchloride, mesylanhydride, tosylanhydride, trifluormethylsulfonylanhydride, nona-fluorobutylsulfonylanhydride, (4-bromo-phenyl)sulfonylanhydride, (4-nitro-phenyl)sulfonylanhydride, (2-nitro-phenyl)sulfonylanhydride, (4-isopropyl-phenyl)sulfonylanhydride, (2,4,6-tri-isopropyl-phenyl)sulfonylanhydride, (2,4,6-trimethyl-phenyl)sulfonylanhydride, (4-tertbutyl-phenyl)sulfonylanhydride, (4-methoxy-phenyl)sulfonylanhydride etc. An additional method which is applicable to the synthesis of those alkyl chains R1 in formula I-III which are interrupted by 1 or 2 oxygen atoms comprises the alkylation of hydroxy compounds by suitable bis(arylsulfonates) or bis(alkylsulfonates)bis(tosylates) and the like, e.g. bis(tosylates) TsO—(CH₂)n-OTs.

The synthesis of hydroxy compounds as starting materials for tosylates, brosylates, nosylates, mesylates, triflates, nonaflates etc. comprisesthe deprotection of OH-protecting groups. As one of the very versatile protecting groups might be mentioned the acetyl protecting group. Many others are known in the art, see e.g. T. W. Greene and P. G. M. Wuts, Protective Groups in Organic Synthesis, 3rd ed, 1999, John Wiley & Sons, pp 17ff).

The hydroxy compounds can alternatively be synthesized directly by those skilled in the art by e.g. hydroboration of corresponding vinylic compounds, reduction of carbonyl compounds, or alkylation of deprotonated homoglutamate derivatives with epoxides (R. C. Larock, Comprehensive Organic Transformations, VCH Publishers 1989, p. 479-582) or by direct β-oxidation of carbonyl compounds via sulfonyloxaziridines (F. A. Davis et al., J. Org. Chem. 1984, 49(17), 3241-3243) or MoOPH (J. Marin et al., JOC 2002, 67, 8440-8449) e.g. at C5.

L-Glutamic acid derivatives are referred to as (2S)-glutamic acid derivatives and D-glutamic acid derivatives are referred to as (2R)-glutamic acid derivatives.

Compounds of general formula IId can be synthesized as described in the examples 1, 2, 3 and 4 if instead of (2S) glutamic acid derivatives the corresponding (2R) glutamic acid derivatives are used.

Compounds of general formula IIc can be synthesized as described in the example 12 if instead of (2S) glutamic acid derivative the corresponding (2R) glutamic acid derivative is used.

Compounds of general formula Ic and compounds of general formula Id can be synthesized identical to the methods described in the examples 5, 6, 7, 8, 9, 10, if compounds general formula IIc or compounds of general formula IId are used instead of compounds of formula IIa as described in the examples.

### EXPERIMENTAL PART

### General Remarks

The composition of “potassium carbonate/kryptofix solution” described in the Examples 6, 7, 8, 9, 13 is 1 mg potassium carbonate and 5 mg kryptofix in 950 μL acetonitrile and 50 μL water. 1 mg (7.24 μmol) potassium carbonate is added to the reaction if 1 mL of the solution is used (Example 6 and Example 7). 1.5 mg (10.9 μmol) potassium carbonate are added to the reaction if 1.5 mL of the solution are used (Example 8 and Example 9).

### Analytical Methods for F-18 Labeled Compounds

F-18 labeled compounds described in the examples herein have been analyzed by radio-TLC (thin-layer-chromatography) and HPLC.

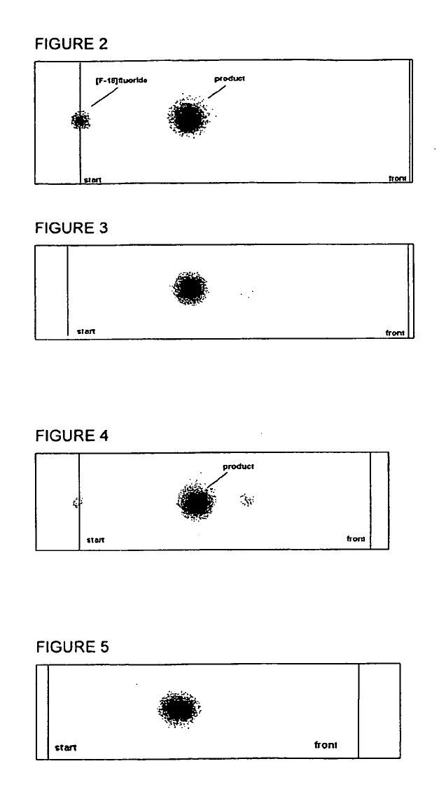

Radio-TLC have been performed using silica plates (Si 60F₂₅₄, Merck) and a solvent systems consisting of n-butanol/acetic acid/water/ethanol (12/3/5/1.5). For examples see FIGS. 2, 3, 4, 5.

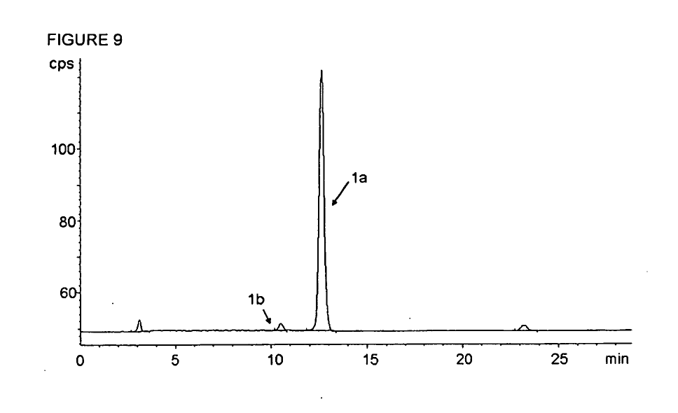

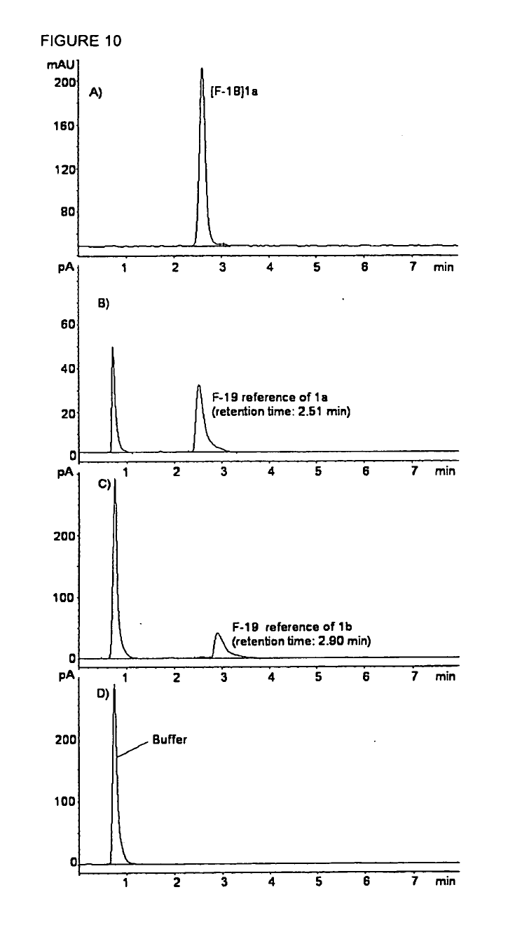

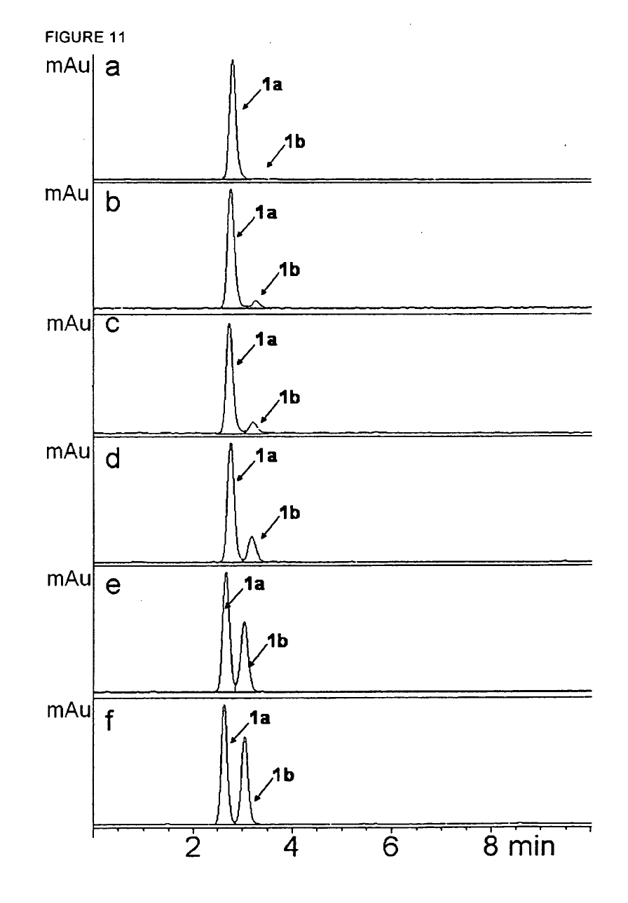

HPLC analysis have been performed using a Hypercarb column (100*4.6 mm, 7μ, Thermo Scientific) with 2% acetonitrile in water+0.1% TFA. For examples see FIG. 9. Co-elution of the radiolabeled compounds with the corresponding F-19 reference compounds was monitored using a Corona detector (ESA Biosciences). For examples see FIGS. 10, 11.

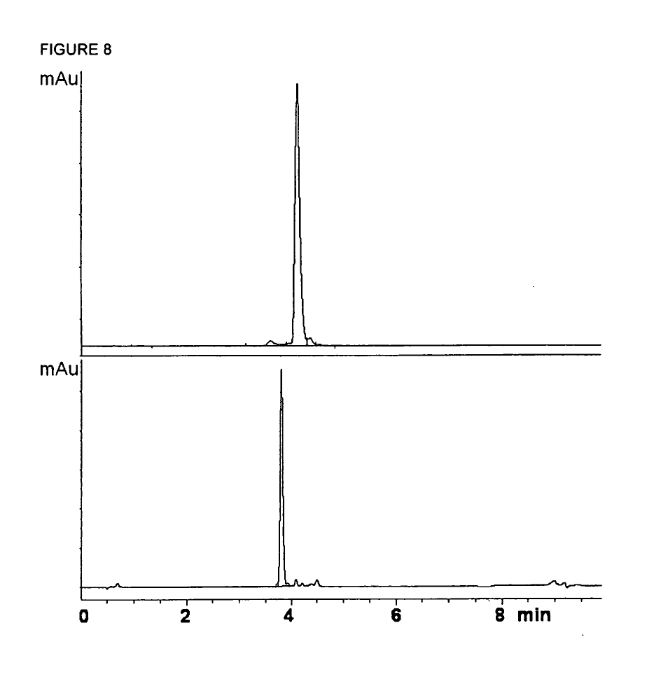

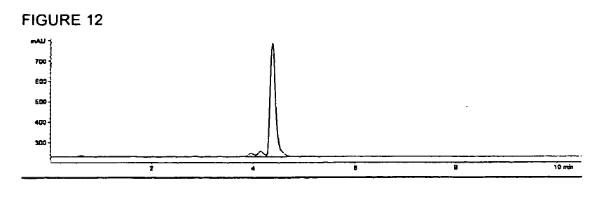

HPLC analysis have been also performed using pre-column derivatization HPLC. 10 μL sample were mixed with 30 μL OPA-reagent (Fluoraldehyde™ o-Phthalaldehyde Reagent Solution; Thermo Scientific). Mixing was done manually or be means of the autosampler of the HPLC. The derivatized sample was analyzed on a C18 column:
Luna (5μ C18(2), 250*4.6 mm, 5μ, Phenomenex); 12% acetonitrile in 10 mM phosphate buffer (pH 7.4); 1.2 mL/min; (For example see FIG. 9) or:
Chromolith (Speed ROD, 50*4.6 mm, 5 μm, Merck); 0-95% acetonitrile in 10 mM phosphate buffer (pH 7.4); 2 mL/min (For examples see FIGS. 8 and 12).

Co-elution of the radiolabeled compounds with the corresponding F-19 reference compounds was monitored using at 340 nM (characteristic wave length of “OPA-derivatives”). For example see FIG. 8).

### Example 1

### Dimethyl (2S,4S)—N-(tert-butoxycarbonyl)-4-[3-(mesyloxy)propyl]-glutamate

### a) Dimethyl (2S,4S)-4-allyl-N-(tert-butoxycarbonyl)-glutamate

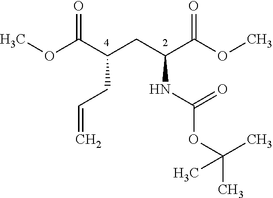 [SMILES: C=CC[C@@H](C[C@H](NC(=O)OC(C)(C)C)C(=O)OC)C(=O)OC]

11.01 g (40 mmol) of dimethyl Boc-glutamate (Advanced Chemtech) were dissolved in 160 mL of tetrahydrofuran and cooled to −70° C. 88 mL (88 mmol) of a 1M solution of lithium bis(trimethylsilyl)amide in tetrahydrofuran were added dropwise at this temperature over a period of one hour, and the mixture was stirred at −70° C. for another 2 hours. 14.52 g (120 mmol) of allylbromide were then added dropwise, and after 2 h at this temperature, the cooling bath was removed and 200 mL of 2N aqueous hydrochloric acid and 400 mL of ethyl acetate were added. The organic phase was separated off, washed with water until neutral, dried over sodium sulphate and filtered, and the filtrate was concentrated. The crude product obtained in this manner was chromatographed in silica gel using a hexane/ethyl acetate gradient, and the appropriate fractions were combined and concentrated.

Yield: 3.3 g (26%)

MS (ESIpos): m/z=316 [M+H]+

1H-NMR (400 MHz, CHLOROFORM-d): Shift [ppm]=1.44 (s, 9H), 1.99-2.02 (m, 2H), 2.31-2.39 (m, 2H), 2.56-2.61 (m, 1H), 3.67 (s, 3H), 3.73 (s, 3H), 4.33-4.15 (m, 1H), 4.33-4.37 (m, 1H), 4.95-4.97 (m, 1H), 5.04-5.10 (m, 2H), 5.67-5.76 (m, 1H).

### b) Dimethyl (2S,4S)—N-(tert-butoxycarbonyl)-4-(3-hydroxypropyl)-glutamate

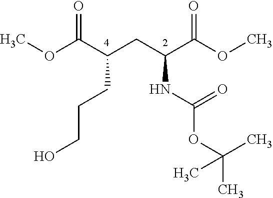 [SMILES: COC(=O)[C@@H](CCCO)C[C@H](NC(=O)OC(C)(C)C)C(=O)OC]

3.15 g (10 mmol) of dimethyl (2S,4S)-4-allyl-N-(tert-butoxycarbonyl)-glutamate were dissolved in 50 mL of tetrahydrofuran and cooled in an ice-bath. Over a period of 20 minutes, 13.3 mL of 1 M diboran/tetrahydrofuran complex in tetrahydrofuran were added dropwise with ice-cooling and under nitrogen, and the mixture was stirred on ice for 1 h and at room temperature overnight. 15 mL of 1 N aqueous sodium hydroxide solution and 13.3 mL of 30% aqueous hydrogen peroxide solution were then added dropwise. After 30 minutes, the mixture was diluted with water, the tetrahydrofuran was distilled off and the remaining aqueous solution was extracted with ethyl acetate. The organic phase to was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated.

Yield: 0.6 g (18%)

MS (ESIpos): m/z=334 [M+H]+

1H-NMR (600 MHz, CHLOROFORM-d): Shift [ppm]=1.44 (s, 9H), 1.47-1.98 (m, 6H), 2.51-2.55 (m, 1H), 3.61-3.62 (m, 2H), 3.68 (s, 3H), 3.74 (s, 3H), 4.37-4.41 (m, 1H), 5.04 (d, 1H).

### c) Dimethyl (2S,4S)—N-(tert-butoxycarbonyl)-4-[3-(mesyloxy)propy]-glutamate

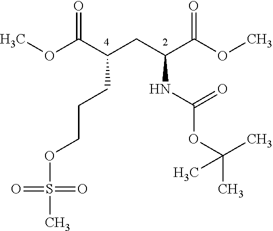 [SMILES: COC(=O)[C@@H](CCCOS(C)(=O)=O)C[C@H](NC(=O)OC(C)(C)C)C(=O)OC]

0.17 g (0.5 mmol) of dimethyl (2S,4S)—N-(tert-butoxycarbonyl)-4-(3-hydroxypropyl)-glutamate was dissolved in dichloromethane and cooled in an ice-bath. After addition of 0.30 g (3 mmol) of triethylamine and 115 mg (1 mmol) of methanesulphonyl chloride, the mixture was stirred on ice for 2 h and then concentrated. The crude product obtained in this manner was chromatographed on silica gel using a hexane/ethyl acetate gradient and the appropriate fractions were combined and concentrated.

Yield: 145 mg (70.5%)

MS (ESIpos): m/z=412 [M+H]+

1H-NMR (300 MHz, CHLOROFORM-d): Shift [ppm]=1.44 (s, 9H), 1.68-1.79 (m, 4H), 1.98-2.05 (m, 2H), 2.48-2.56 (m, 1H), 3.02 (s, 3H), 3.69 (s, 3H), 3.74 (s, 3H), 4.20-4.24 (m, 2H), 4.30-4.39 (m, 1H), 4.95-4.99 (m, 1H).

### Example 2

### Di-tert-butyl (2S,4S)—N-(tert-butoxycarbonyl)-4-[3-(mesyloxy) propyl]-glutamate

### a) Di-tert-butyl (2S,4S)-4-allyl-N-(tert-butoxycarbonyl)-glutamate

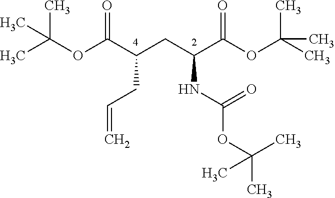 [SMILES: C=CC[C@@H](C[C@H](NC(=O)OC(C)(C)C)C(=O)OC(C)(C)C)C(=O)OC(C)(C)C]

26.96 g (75 mmol) of di-tert-butyl Boc-glutamate (Journal of Peptide Research (2001), 58, 338) were dissolved in 220 mL of tetrahydrofuran (THF) and cooled to −70° C. 165 mL (165 mmol) of a 1M solution of lithium bis(trimethylsilyl)amide in THF were added dropwise over a period of two hours at this temperature and the mixture was stirred at −70° C. for another 2 hours. 27.22 g (225 mmol) of allyl bromide were then added dropwise, and after 2 h at this temperature, the cooling bath was removed and 375 mL of 2N aqueous hydrochloric acid and 1.25 L of ethyl acetate were added. The organic phase was separated off, washed with water until neutral, dried over sodium sulphate and filtered, and the filtrate was concentrated. The crude product obtained in this manner was chromatographed in silica gel using a hexane/ethyl acetate gradient, and the appropriate fractions were combined and concentrated.

Yield: 15.9 g (53.1%)

MS (ESIpos): m/z=400 [M+H]+

¹H NMR (300 MHz, CHLOROFORM-d) d ppm 1.32-1.58 (m, 27H) 1.81-1.92 (m, 2H) 2.25-2.39 (m, 2H) 2.40-2.48 (m, 1H), 4.10-4.18 (m, 1H) 4.85-4.92 (d, 1H) 5.02-5.11 (m, 2H) 5.68-5.77 (m, 1H)

### b) Di-tert-butyl (2S,4S)—N-(tert-butoxycarbonyl)-4-(3-hydroxypropyl)-glutamate

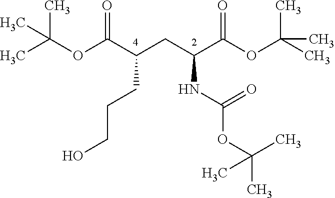 [SMILES: CC(C)(C)OC(=O)N[C@@H](C[C@H](CCCO)C(=O)OC(C)(C)C)C(=O)OC(C)(C)C]

15.58 g (39 mmol) of di-tert-butyl (2S,4S)-4-allyl-N-(tert-butoxycarbonyl)-glutamate were dissolved in 200 mL of tetrahydrofuran and cooled in an ice-bath. Over a period of about 20 minutes, 54.6 mL (54.6 mmol) of 1 M diboran/tetrahydrofuran complex in tetrahydrofuran were added dropwise with ice-cooling and under nitrogen, and the mixture was stirred on ice for 2 h and at room temperature overnight. It was cooled again to 0° C. and 58.5 mL of 1 N aqueous sodium hydroxide solution and 58.5 mL of 30% aqueous hydrogen peroxide solution were then added dropwise. After 30 minutes, the mixture was diluted with water, the tetrahydrofuran was distilled off and the remaining aqueous solution was extracted with ethyl acetate. The organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated.

Yield: 8.5 g (52%)

MS (ESIpos): m/z=418 [M+H]+

1H NMR (300 MHz, CHLOROFORM-d) d ppm 1.32-1.58 (m, 27H) 1.60-1.70 (m, 2H) 1.73-1.94 (m, 4H) 2.05-2.12 (m, 1H), 2.33-2.40 (m, 1H) 3.58-3.68 (m, 2H) 4.15-4.22 (m, 1H) 4.95-5.03 (d, 1H)

### c) Di-tert-butyl (2S,4S)—N-(tert-butoxycarbonyl)-4-[3-(mesyloxy])propyl]-glutamate

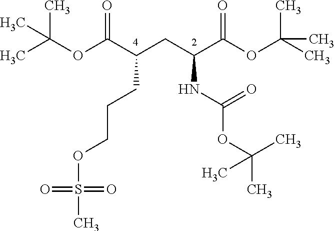 [SMILES: CC(C)(C)OC(=O)N[C@@H](C[C@H](CCCOS(C)(=O)=O)C(=O)OC(C)(C)C)C(=O)OC(C)(C)C]

418 mg (1 mmol) of di-tert-butyl (2S,4S)—N-(tert-butoxycarbonyl)-4-(3-hydroxypropyl)-glutamate were dissolved in 20 mL of dichloromethane and cooled in an ice-bath. After addition of 0.83 mL (6 mmol) of triethylamine and 229 mg (2 mmol) of methanesulphonyl chloride, the mixture was stirred on ice for 2 h and then concentrated. The crude product obtained in this manner was chromatographed on silica gel using a hexane/ethyl acetate gradient and the appropriate fractions were combined and concentrated.

Yield: 350 mg (70.6%)

MS (ESIpos): m/z=496 [M+H]+

1H-NMR (300 MHz, CHLOROFORM-d): Shift [ppm]=1.44-147 (m, 27H), 1.61-1.96 (m, 6H), 2.32-2.41 (q, 1H), 3.02 (s, 3H), 4.11-4.18 (m, 2H), 4.88-4.91 (d, 1H).

### Example 3

### Di-tert-butyl (2S,4S)—N-(tert-butoxycarbonyl)-4-[3-(tosyloxy)propy]-glutamate

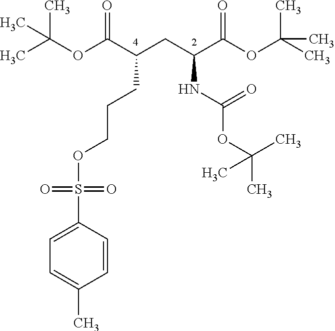 [SMILES: Cc1ccc(S(=O)(=O)OCCC[C@@H](C[C@H](NC(=O)OC(C)(C)C)C(=O)OC(C)(C)C)C(=O)OC(C)(C)C)cc1]

418 mg (1 mmol) of di-tert-butyl (2S,4S)—N-(tert-butoxycarbonyl)-4-(3-hydroxypropyl)-glutamate were dissolved in 20 mL of dichloromethane and cooled in an ice-bath. After addition of 0.61 g (6 mmol) of triethylamine and 0.38 g (2 mmol) p-toluenesulphonyl chloride, the mixture was stirred on ice for 2 h, overnight at room temperature and then concentrated. The crude product obtained in this manner was chromatographed on silica gel using a hexane/ethyl acetate gradient and the appropriate fractions were combined and concentrated.

Yield: 0.37 g (64.7%)

MS (ESIpos): m/z=572 [M+H]+

1H NMR (300 MHz, CHLOROFORM-d) d ppm 1.37-1.93 (m, 33H) 2.18-2.35 (m, 4H) 4.01-4.16 (m, 3H) 4.84 (d, 1H) 7.35 (d, 2H) 7.78 (d, 2H)

### Example 4

### Di-tert-butyl (2S,4S)—N-(tert-butoxycarbonyl)-4-(3-{[(4-nitrophenyl)-sulfonyl]oxy}propyl)-glutamate

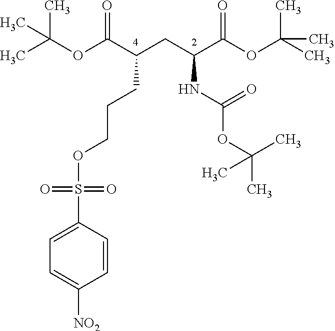 [SMILES: CC(C)(C)OC(=O)N[C@@H](C[C@H](CCCOS(=O)(=O)c1ccc([N+](=O)[O-])cc1)C(=O)OC(C)(C)C)C(=O)OC(C)(C)C]

5.22 g (12.5 mmol) of di-tert-butyl (2S,4S)—N-(tert-butoxycarbonyl)-4-(3-hydroxypropyl)-glutamate were dissolved in 125 mL of dichloromethane and cooled in an ice-bath. After addition of 7.59 g (75 mmol) of triethylamine and 5.54 g (25 mmol) nitrophenylsulfonyl chloride, the mixture was stirred on ice for 2 h and then concentrated. The crude product obtained in this manner was chromatographed on silica gel using a hexane/ethyl acetate gradient and the appropriate fractions were combined and concentrated.

Yield: 4.7 g (62.4%)

MS (ESIpos): m/z=603 [M+H]+

1H NMR (300 MHz, CHLOROFORM-d) d ppm 1.42-1.45 (m, 27H) 1.57-1.87 (m, 6H) 2.29 (m, 1H) 4.01 (m, 1H) 4.13-4.16 (m, 2H) 4.86 (d, 1H) 8.12 (d, 2H) 8.42 (d, 2H)

### Example 5

### One-pot synthesis of (2S,4S)-4-(3-[¹⁸]Fluoropropyl)-glutamic acid starting from Dimethyl (2S,4S)—N-(tert-butoxycarbonyl)-4-[3-(mesyloxy)propy]-glutamate using a “Eckert&Ziegler modular lab” synthesizer

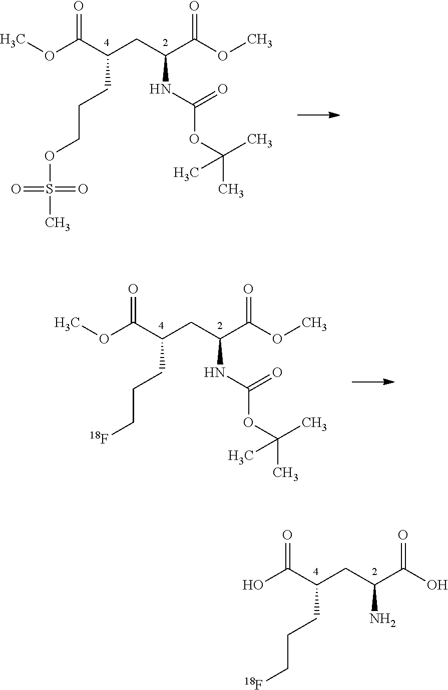 [SMILES: COC(=O)[C@@H](CCCOS(C)(=O)=O)C[C@H](NC(=O)OC(C)(C)C)C(=O)OC ; COC(=O)[C@@H](CCC[18F])C[C@H](NC(=O)OC(C)(C)C)C(=O)OC ; N[C@@H](C[C@H](CCC[18F])C(=O)O)C(=O)O]

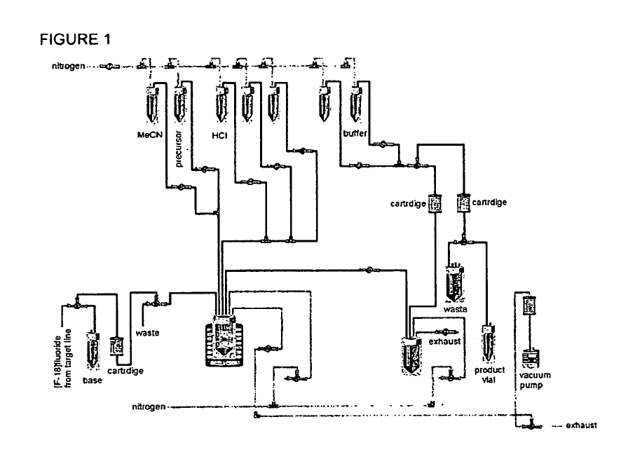

The syntheses were performed on a remote controlled synthesizer, “Eckert&Ziegler modular lab” (FIG. 1).

a) Purification using Hilic SPE

[F-18]fluoride (29.6 GBq) was trapped on a QMA cartridge (Waters, SepPak light). The activity was eluted with 1 mL kryptofix/potassium carbonate solution (acetonitrile/water) into the reaction vessel. The mixture was dried (120° C., nitrogen stream, vacuum). Drying was repeated after addition of 1 ml acetonitrile. 5 mg mesylate precursor in 1 mL acetonitrile were added to the dried residue and the resulting solution was stirred at 110° C. (displayed reactor temperature) for 10 min. The solvent was evaporated (110° C., nitrogen stream) and 1 mL 4N HCl was added.

The mixture was heated at 150° C. for 5 min. After cooling to 60° C., the solution was diluted with acetonitrile (80 mL) and passed through a Hilic cartridge (ZIC-Hilic SPE, 3 ml, 1 g, SeQuant). The cartridge was dried by nitrogen steam, washed with ethanol (3 mL) and dried with stream of nitrogen. (2S,4S)-4-{3-[F-18]fluoropropyl}-glutamic acid was eluted with 10 mL buffer (70 mg Na₂HPO₄ 2H₂O, 60 mg NaCl in 10 mL water) into the product vial.

Radiochemical yield: 3.6 GBq (19% decay corrected (d.c.))

Overall process time: 61 min

Radiochemical purity: 90% (determined by TLC, FIG. 2)

b) Purification using MCX SPE

[F-18]fluoride (24.6 GBq) was trapped on a QMA cartridge (Waters, SepPak light). The activity was eluted with 1 mL kryptofix/potassium carbonate solution (acetonitrile/water) into the reaction vessel. The mixture was dried (120° C., nitrogen stream, vacuum). Drying was repeated after addition of 1 ml acetonitrile. 5 mg mesylate precursor in 1 mL acetonitrile were added to the dried residue and the resulting solution was stirred at 110° C. for 10 min. The solvent was evaporated (110° C., nitrogen stream) and 1 mL 4N HCl was added. The mixture was heated at 150° C. for 5 min. After cooling to 60° C., the solution was diluted with water (3 mL) and passed through a MCX cartridge (Oasis MCX 20 cc (1 g), Waters). The cartridge was washed with 1N HCl (4 mL) and ethanol (5 mL) and (2S,4S)-4-{3-(F-18]fluoropropyl}-glutamic acid was eluted with 5 mL buffer (sodium bicarbonate) into the product vial.

Radiochemical yield: 4.6 GBq (28% d.c.)

Overall process time: 64 min

Radiochemical purity: 95% (determined by TLC, FIG. 3)

### Example 6

### One-pot synthesis of (2S,4S)-4-(3-[¹⁸]Fluoropropyl)-glutamic acid starting from Di-tert-butyl (2S,4S)—N-(tert-butoxycarbonyl)-4-(3-(mesyloxy])propy]-glutamate using a “Eckert&Ziegler modular lab” synthesizer

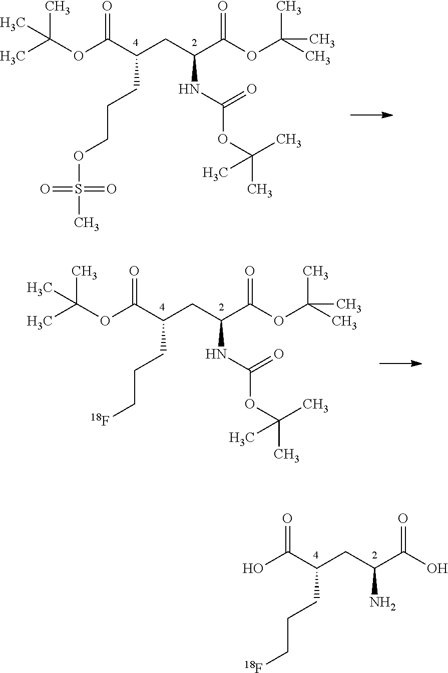

The synthesis were performed on a remote controlled synthesizer, “Eckert&Ziegler modular lab” (FIG. 1).

[F-18]fluoride (32.2 GBq) was trapped on a QMA cartridge (Waters, SepPak light). The activity was eluted with 1 mL kryptofix/potassium carbonate solution (acetonitrile/water) into the reaction vessel. The mixture was dried (120° C., nitrogen stream, vacuum). Drying was repeated after addition of 1 ml acetonitrile. 5 mg mesylate precursor in 1 mL acetonitrile were added to the dried residue and the resulting solution was stirred at 110° C. for 10 min. The solvent was evaporated (110° C., nitrogen stream) and 1 mL 4N HCl was added.

The mixture was heated at 120° C. for 10 min. After cooling to 60° C., the solution was diluted with water (3 mL) and passed through a MCX cartridge (Oasis MCX 20 cc (1 g), Waters). The cartridge was washed with 0.1N HCl (4 mL) and ethanol (5 mL) and (2S,4S)-4-{3-[F-18]fluoropropyl}-glutamic acid was eluted with 5 mL buffer (sodium bicarbonate) into the product vial.

Radiochemical yield: 2.3 GBq (11% d.c.)

Overall process time: 60 min

Radiochemical purity: 90% (determined by TLC, FIG. 4)

### Example 7

### One-pot synthesis of (2S,4S)-4-(3-[¹⁸F]Fluoropropyl)-glutamic acid starting from Di-tert-butyl (2S,4S)—N-(tert-butoxycarbonyl)-4-[3-(tosyloxy)propy]-glutamate using a “Eckert&Ziegler modular lab” synthesizer

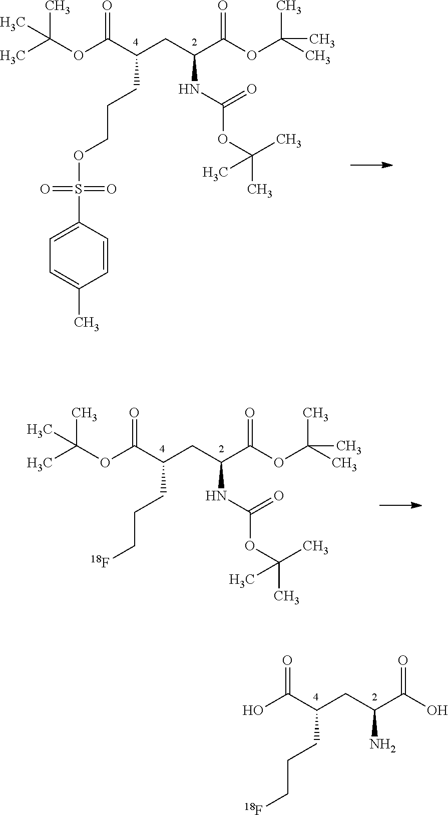

The synthesis were performed on a remote controlled synthesizer, “Eckert&Ziegler modular lab” (FIG. 1).

[F-18]fluoride (22.9 GBq) was trapped on a QMA cartridge (Waters, SepPak light). The activity was eluted with 1 mL kryptofix/potassium carbonate solution (acetonitrile/water) into the reaction vessel. The mixture was dried (120° C., nitrogen stream, vacuum). Drying was repeated after addition of 1 ml acetonitrile. 5 mg tosylate precursor in 1 mL acetonitrile were added to the dried residue and the resulting solution was stirred at 100° C. for 10 min. The solvent was evaporated (110° C., nitrogen stream) and 1 mL 2N HCl was added.

The mixture was heated at 120° C. for 10 min. After cooling to 60° C., the solution was diluted with water (3 mL) and passed through a MCX cartridge (Oasis MCX 20 cc (1 g), Waters). The cartridge was washed with 0.1N HCl (4 mL) and ethanol (5 mL) and (2S,4S)-4-{3-[F-18]fluoropropyl}-glutamic acid was eluted with 3 mL buffer (sodium bicarbonate) into the product vial.

Radiochemical yield: 3.6 GBq (26% d.c.)

Overall process time: 57 min

Radiochemical purity: 99% (determined by TLC, FIG. 5)

### Example 8

### One-pot synthesis of (2S,4S)-4-(3-[(¹⁸F]Fluoropropyl)-glutamic acid starting from Di-tert-butyl (2S,4S)—N-(tert-butoxycarbonyl)-4-(3-{[(4-nitrophenyl)sulfonyl]oxy}propyl)-glutamate using a “Eckert&Ziegler modular lab” synthesizer (FIG. 1)

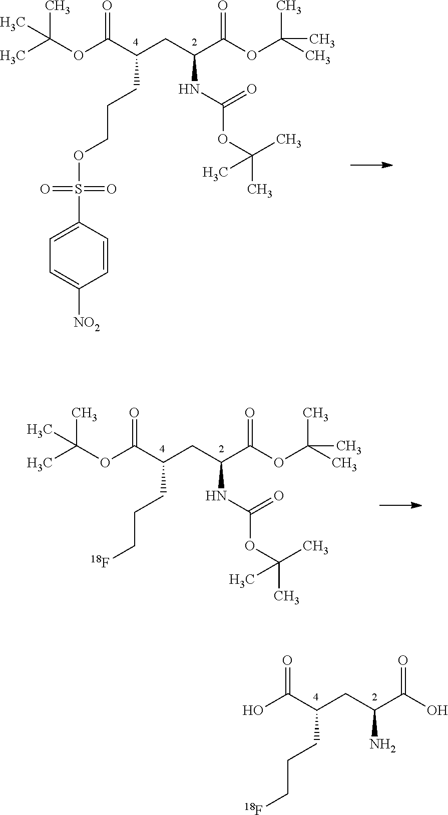

The syntheses were performed on a remote controlled synthesizer, “Eckert&Ziegler modular lab” (FIG. 1).

### a) Fluorination Using [F-18]Fluoride/Kryptofix/Potassium Carbonate

[F-18]fluoride (see Table 1 for details) was trapped on a QMA cartridge (Waters, SepPak light). The activity was eluted with 1.5 mL kryptofix/potassium carbonate solution (acetonitrile/water) into the reaction vessel. The mixture was dried (120° C., nitrogen stream, vacuum). Drying was repeated after addition of 1 ml acetonitrile. 5 mg nosylate precursor in 1 mL acetonitrile were added to the dried residue and the resulting solution was stirred (for reaction time and temperature see Table 1). 2N HCl (2 mL) was added without previous evaporation and the mixture was heated at 100° C. for 5 min. After cooling to 60° C., the solution was diluted with water (50 mL) and passed through a HR-P (Chromafix HR-P, Macherey-Nagel) and a MCX cartridge (Oasis MCX 20 cc (1 g), Waters). The MCX cartridge was washed with saline (20 mL) and (2S,4S)-4-{3-[F-18]fluoropropyl}-glutamic acid was eluted with 10 mL buffer (70 mg Na₂HPO₄2H₂O, 60 mg NaCl in 10 mL water) into the product vial. Results are summarized in Table 1.

TABLE 1
Results conversion of nosylate precursor

| | reaction | | radio- | overall | | Diastereo- |
|---|---|---|---|---|---|---|
| | tem- | reaction | chem. | process. | radiochem. | meric |
| entry | perature | time | yield | time | purity (TLC) | purity* |
| 1 | 80° C. | 10 min | 1.0 GBq | 46 min | 94% | 92% |
| | | | (73% d.c.) | | | |
| 2 | 80° C. | 5 min | 1.2 GBq | 44 min | 97% | 95% |
| | | | (61% d.c.) | | | |
| 3 | 80° C. | 1 min | 2.0 GBq | 41 min | 97% | 97% |
| | | | (63% d.c.) | | | |
| 4 | 60° C. | 5 min | 3.5 GBq | 44 min | 98% | 97% |
| | | | (56% d.c.) | | | |
| 5 | 60° C. | 1 min | 6.3 GBq | 41 min | 98% | 98% |
| | | | (67% d.c.) | | | |

*determined by HPLC.

Radiochemical purity and diastereomeric ratio was determined by pre-column derivatization HPLC (FIG. 8).

### b) Fluorination Using [F-18]Fluoride/Tetrabutyl Ammonium Bicarbonate

[F-18]Fluoride solution was passed through on a column packed with DOWEX 1×8-200 (40 mg) which was subsequently flushed with inert gas. [F-18]Fluoride was eluted from the column with an aqueous solution of tetrabutylammonium bicarbonate (n-Bu₄NHCO₃) in water/acetonitrile into the reactor vessel. The mixture was dried by heating under a stream of inert gas. The drying procedure was repeated after addition of 1 ml acetonitrile.

5 mg noslyate precursor in 1 mL acetonitrile were transferred into the reactor vessel containing the dried tetrabutylammonium [F-18]Fluoride. The mixture was heated at 80° C. for 10 min. After cooling, 2N HCl (1 mL) was added and the mixture was heated at 100° C. Subsequently, 2N NaOH (1.35 mL) was added followed by heating at 80° C. The reaction mixture was transferred into a mixing vessel (filled with 50 mL 0.1NHCl). The reactor vessel was washed with water into the mixing vessel. The water solution containing the radiolabeled product was passed through a HR-P cartridge onto two MCX plus (SepPak plus, waters) cartridges. After the MCX cartridges are washed with 20 mL isotonic saline, residual saline was removed with argon. Finally, the product was eluted from the two cartridges with 20 mL isotonic buffer 140 mg Na₂HPO₄.2H₂O and 120 mg NaCl in 20 mL water) into the product vail.

Radiochemical yield: 12.9 GBq (40% d.c.)

Radiochemical purity: 97%

Isomeric purity: >98%

### c) Fluorination Using [F-18]Fluoride/Tetrabutyl Ammonium Bicarbonate

[F-18]Fluoride solution was passed through a QMA cartridge (QMA light, Waters) which was subsequently flushed with inert gas. [F-18]Fluoride was to eluted from the column with an aqueous solution of tetrabutylammonium bicarbonate (n-Bu₄NHCO₃) in water/acetonitrile into the reactor vessel. The mixture was dried by heating under a stream of inert gas. The drying procedure was repeated after addition of 1 ml acetonitrile.

5 mg noslyate precursor in 1 mL acetonitrile were transferred into the reactor vessel containing the dried tetrabutylammonium [F-18]Fluoride. The mixture was heated at 80° C. for 700 s. After cooling, 2N HCl (1 mL) was added and the mixture was heated at 100° C. Subsequently, 2N NaOH (1.35 mL) was added followed by heating at 80° C. The reaction mixture was transferred into a mixing vessel (filled with 50 mL 0.1 NHCl). The reactor vessel was washed with water into the mixing vessel. The water solution containing the radiolabeled product was passed through a HR-P cartridge onto two MCX plus (SepPak plus, waters) cartridges. After the MCX cartridges are washed with 20 mL isotonic saline, residual saline was removed with argon. Finally, the product was eluted from the two cartridges with 20 mL isotonic buffer 140 mg Na₂HPO₄.2H₂O and 120 mg NaCl in 20 mL water) into the product vail.

Radiochemical yield: 40±6.4% (corrected for decay)

Radiochemical purity: 92.4±2.3% %

Diastereomeric ratio: >98/2

Radiochemical purity and diastereomeric ratio was determined by pre-column derivatization HPLC (FIG. 9).

### Example 9

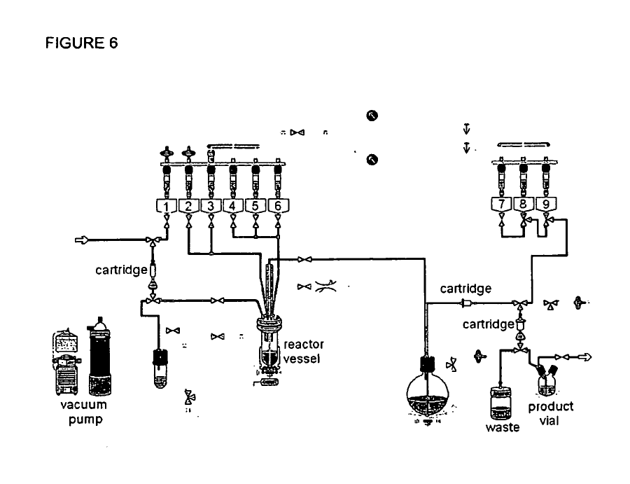

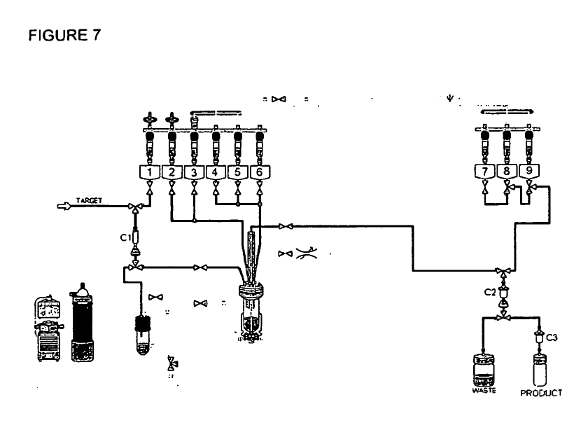

### One-pot synthesis of (2S,4S)-4-(3-[¹⁸F]Fluoropropyl)-glutamic acid starting from Di-tert-butyl (2S,4S)—N-(tert-butoxycarbonyl)-4-(3-{([(4-nitrophenyl)sulfonyl]oxy}propyl)-glutamate using a “GE tracerlab FX” synthesizer (FIG. 6)

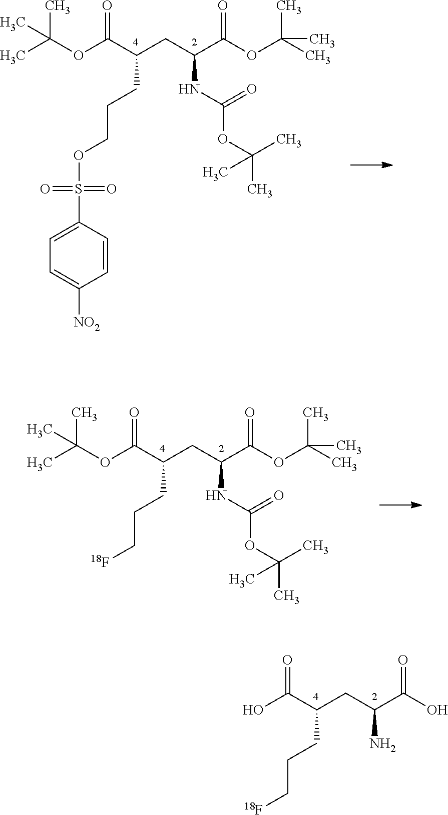

[F-18]fluoride (1.6 GBq) was trapped on a QMA cartridge (Waters, SepPak light). The activity was eluted with 1.5 mL kryptofix/potassium carbonate solution (acetonitrile/water) into the reaction vessel. The mixture was dried (120° C., nitrogen stream, vacuum). Drying was repeated after addition of 1 ml acetonitrile. 5 mg nosylate precursor in 1 mL acetonitrile were added to the dried residue and the resulting solution was stirred at 60° C. for 10 min. 2N HCl (2 mL) was added without previous evaporation and the mixture was heated at 110° C. for 5 min. After cooling to 60° C., the solution was diluted with water (50 mL) and passed through a HR-P (Chromafix HR-P, Macherey-Nagel) and a MCX cartridge (Oasis MCX 20 cc (1 g), Waters). The MCX cartridge was washed with saline (10 mL) and (2S,4S)-4-{3-[F-18]fluoropropyl}-glutamic acid was eluted with 10 mL buffer (70 mg Na₂HPO₄2H₂O, 60 mg NaCl in 10 mL water) into the product vial.

Radiochemical yield: 0.46 GBq (39% d.c.)

Overall process time: 51 min

Radiochemical purity: 95% (determined by TLC)

### Example 10

### Synthesis of (2S,4S)-4-(3-[¹⁸F]Fluoropropyl)-glutamic acid starting from Di-tert-butyl (2S,4S)—N-(tert-butoxycarbonyl)-4-(3-{[(4-nitrophenyl)sulfonyl]oxy}propyl)-glutamate using a “GE tracerlab MX” synthesizer

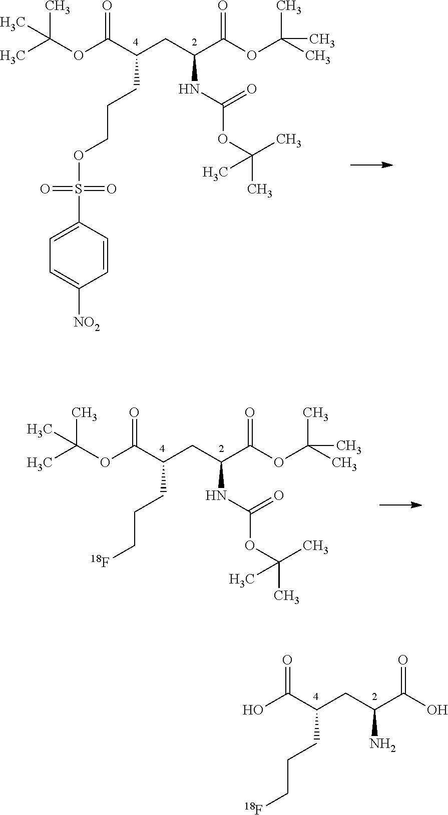

### a) For Synthesis of on the Cassette Type Synthesizer “GE Tracerlab MX” a Kit was Assembled

A commercially available FDG cassette (GE) was adopted:
tC18 and alumina N cartridges were removed and the second and third manifold were connected with a tube,
the “green spike” (originally used for the “buffer vial” of the FOG process was removed).

The reagent kit for the (2S,4S)-4-(3-[¹⁸F]fluoropropyl)-glutamic acid synthesis process included:
A preconditioned QMA cartridge (Waters, ABX),
A “eluent vial”: potassium carbonate, kryptofix in 300 μL MeCN and 300 μL water (for detailed composition see Table 2 and the description within passage “c” of the present example),
A blue capped vial, filled with 8 mL MeCN (to be placed on valve “3” of the modified FDG cassette),
Two empty 30 mL syringes,
A red capped “precursor vial”, filled with a solution of Di-tert-butyl (2S,4S)—N-(tert-butoxycarbonyl)-4-(3-{[(4-nitrophenyl)sulfonyl]oxy}propyl)-glutamate in 1.5 ml MeCN (to be placed on valve “5” of the modified FDG cassette),
A “water bag” (filled with 100 or 250 mL water for injectable solutions),
A yellow capped “HCl vial”, filled with 2 ml 2M HCl (to be placed on valve “8” of the modified FDG cassette),
A 20 mL syringe filled the formulation buffer (140 mg Na₂HPO₄2H₂O, 120 mg NaCl dissolved in 20 mL water for injectable solutions, to be placed on valve “9” of the modified FDG cassette,
3 MCX plus (Waters) cartridges (to be placed between valves 12 and 13 of the modified FDG cassette),
A 500 mg Hypercarb cartridge (Thermo Fischer, to be placed between valve 11 of the modified FDG cassette and the product vial),
A sterile product vial (20 mL or 30 mL),
A needle and a sterile filter,
A vent needle and a sterile vent filter.

### b) Radiosynthesis Using Tracerlab MX, Investigation of Influence of Temperature and Base/Precursor Ratio

The synthesis of (2S,4S)-4-(3-[¹⁸F]fluoropropyl)-glutamic acid was performed using an adapted sequence program, wherein:
The “acetonitrile vial”, the “precursor via” and the “HCl vial” were pressurized,
[F-18] fluoride was transferred to the Tracerlab MX and loaded onto the QMA cartridge,
The activity was transferred using the solution in the “Eluent vial” into the reactor.
The mixture was dried at 95% using nitrogen stream, vacuum and the addition of acetonitrile portions,
The solution of the “precursor vial” was transferred to the reactor (resulting in 4.5±0.5 mg precursor in the reactor) and the resulting mixture was heated for 5 min (for “reaction temperature” see Table 2 and description within passage “c” of the present example,
HCl from the “HCl vial” was transferred to the reactor vial using the 30 mL syringes and the mixture was heated at 120° C. for 10 min,
The crude product mixture was diluted in the left 30 mL syringe with water (from “water bag”) and passed through the MCX cartridges into the waste bottle,
The MCX cartridges were washed with 30 mL water (from “water bag”),
The formulation buffer solution was transferred from the 20 ml syringe into the 30 ml syringe and subsequently passed through the MCX cartridges and the Hypercarb cartridge into the product vial.

The overall synthesis time of the process was 34-35 min.

The influence of reaction temperature and composition of the “eluent vial” was investigated, the results are summarized in Table 2. No epimerization was found using base/precursor ratios below 1 (entry 1-8), whereas low reaction temperatures are needed to prevent significant epimerization at base/precursor ratio greater than 1 (entry 9, 12, 15).

TABLE 2
Results of conversion of nosylate precursor

| | re- | | Ratio of | | |
|---|---|---|---|---|---|
| | action | | K₂CO₃/ | radio- | diastereo- |
| | temper- | Composition | labeling | chem. | meric |
| entry | ature | of “eluent vial” | precursor | purity^{a} | purity^{b} |
| 1 | 70° C. | 0.5 mg K₂CO₃, | 0.48 | >95% | >98/2 |
| 2 | 90° C. | 2.5 mg kryptofix, | | >95% | >98/2 |
| 3 | 120° C. | 300 μL MeCN, | | >95% | >98/2 |
| | | 300 μL H₂O | | | |
| 4 | 70° C. | 0.75 mg K₂CO₃, | 0.73 | >95% | >98/2 |
| 5 | 120° C. | 3.75 mg kryptofix, | | >95% | >98/2 |
| | | 300 μL MeCN , | | | |
| | | 300 μL H₂O | | | |
| 6 | 70° C. | 1.0 mg K₂CO₃, | 0.97 | >95% | >98/2 |
| 7 | 90° C. | 5.0 mg kryptofix, | | >95% | >98/2 |
| 8 | 120° C. | 300 μL MeCN, | | >95% | >98/2 |
| | | 300 μL H₂O | | | |
| 9 | 70° C. | 1.15 mg K₂CO₃, | 1.11 | >95% | >98/2 |
| 10 | 90° C. | 5.12 mg kryptofix, | | >95% | 95/5 |
| 11 | 120° C. | 300 μL MeCN, | | >95% | 60/40 |
| | | 300 μL H₂O | | | |
| 12 | 70° C. | 1.3 mg K₂CO₃, | 1.26 | >95% | ≈98/2 |
| 13 | 90° C. | 6.5 mg kryptofix, | | >95% | 92/8 |
| 14 | 120° C. | 300 μL MeCN, | | >95% | 60/40 |
| | | 300 μL H₂O | | | |
| 15 | 70° C. | 1.5 mg K₂CO₃, | 1.45 | >95% | ≈95/5 |
| 16 | 90° C. | 7.5 mg kryptofix, | | >95% | 82/18 |
| 17 | 120° C. | 300 μL MeCN, | | >95% | 50/50 |
| | | 300 μL H₂O | | | |
| 18 | 15 | 2.0 mg K₂CO₃, | 1.94 | >95% | ≈95/5 |
| 19 | 90° C. | 10.0 mg kryptofix, | | >95% | 78/22 |
| 20 | 120° C. | 300 μL MeCN, | | >95% | 50/50 |
| | | 300 μL H₂O | | | |

a) determined by TLC and HPLC;
b) given as diastereomeric ratio, determined by HPLC (for examples see FIG 10, 11)

c) Routine Synthesis of (2S,4S)-4-(3-[¹⁸F]fluoropropyl)-Glutamic Acid on Tracerlab MX

A reagent kit as described within passage “a” of the present example was used. The “Eluent vial” was filled with a solution of 1.0 mg K₂CO₃, 5.0 mg kryptofix in 300 μL MeCN and 300 μL H₂O. 1.1-86 GBq [F-18]fluoride (n>20) were transferred the Tracerlab MX. The activity was trapped on the QMA cartridge (QMA light, Waters) and eluted using the mixture in the “Eluent vial” into the reactor vial. The mixture was dried at 95° C. using nitrogen stream and vacuum. Drying was repeated after addition of portions of acetonitrile. The solution of the “Precursor vial” was transferred to the reaction vial to reach a amount of 4.5±0.5 mg Di-tert-butyl (2S,4S)—N-(tert-butoxycarbonyl)-4-(3-{[(4-nitrophenyl)sulfonyl]oxy}propyl)-glutamate in the reactor. The mixture was heated at 70° C. for 5 min. The acid from the “HCl vial” was transferred via the mL syringes into the reaction vial and the mixture was heated at 120° C. for 5 min with open exhaust and 5 min with closed exhaust. In the left 30 mL syringe, the crude product mixture was diluted with water (20 mL) and passed through 3 MCX cartridges (MCX plus, Waters). The cartridges were washed with 30 mL water. The formulation buffer was transferred from the 20 mL syringe at valve 9 into the right 30 mL syringe and passed through the MCX cartridges and the Hypercarb cartridge (500 mg, Thermo Scientific) into the product vial. 0.5-41 GBq (44-56% not corrected for decay) (2S,4S)-4-(3-[¹⁸]-fluoropropyl)-glutamic acid were obtained in 34-35 min synthesis time. The radiochemical purity was determined to be greater than 98% using Radio-TLC; HPLC and Derivatization-HPLC.

### Example 11

### One-pot synthesis of (2S,4S)-4-(3-[¹⁸]fluoropropyl)-glutamic acid starting from Di-tert-butyl (2S,4S)—N-(tert-butoxycarbonyl)-4-(3-{[(4-nitrophenyl)sulfonyl]oxy}propyl)-glutamate using a “GE tracerlab FX” synthesizer (FIG. 7)

[F-18]fluoride (3.97 GBq) was trapped on a QMA cartridge (Waters, SepPak light). The activity was eluted with 1.5 mL kryptofix/potassium carbonate solution (5 mg kryptofix, 1 mg potassium carbonate in 1.25 mL acetonitrile and 0.25 mL water) into the reaction vessel. The mixture was dried (120° C., nitrogen stream, vacuum). Drying was repeated after addition of 1 ml acetonitrile. 5 mg nosylate precursor in 1 mL acetonitrile were added to the dried residue and the resulting solution was stirred at 70° C. for 5 min. 2M HCl (1 mL) was added without previous evaporation and the mixture was heated at 110° C. for 10 min. After cooling to 60° C., the solution was diluted with water (10 mL) and passed through 3 MCX cartridges (MCX plus, Waters). The MCX cartridges were washed with water (2×10 mL) and 15 mL formulation buffer (105 mg Na₂HPO₄ 2H₂O, 90 mg NaCl in 15 mL water) were passed trough the MCX cartridges and a Hypercarb cartridge (500 mg, Thermo Scientific) into the product vial. 1.8 GBq (46% not corrected for decay) (2S,4S)-4-{3-[¹⁸F]fluoropropyl}-glutamic were obtained in 41 min synthesis time. The radiochemical purity and diastereomeric excess was determined to be greater than 98% using Radio-TLC; HPLC and Derivatization-HPLC.

### Example 12

### Di-tert-butyl (2S,4R)—N-(tert-butoxycarbonyl)-4-(3-{[(4-nitrophenyl)-sulfonyl]oxy}Propyl)-glutamate

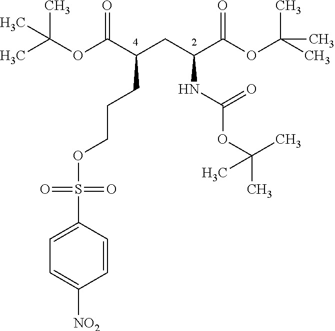 [SMILES: CC(C)(C)OC(=O)N[C@@H](C[C@@H](CCCOS(=O)(=O)c1ccc([N+](=O)[O-])cc1)C(=O)OC(C)(C)C)C(=O)OC(C)(C)C]

### a) Di-tert-butyl (4S)-2-[3-(benzyloxy)-1-hydroxypropyl]-4-[(tert-butoxycarbonyl)-aminopentanedioate

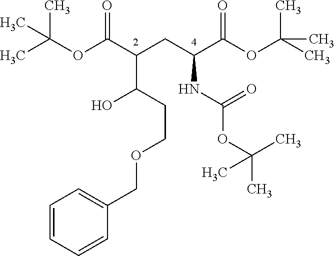 [SMILES: CC(C)(C)OC(=O)N[C@@H](CC(C(=O)OC(C)(C)C)C(O)CCOCc1ccccc1)C(=O)OC(C)(C)C]

5.39 g (15 mmol) of di-tert-butyl Boc glutamate (Journal of Peptide Research (2001), 58, 338) were dissolved in 45 mL of tetrahydrofuran (THF) and cooled to −70° C. 33 mL (33 mmol) of a 1M solution of lithium bis(trimethylsilyl)amide in THF were added dropwise over a period of 45 min at this temperature and the mixture was stirred at −70° C. for another 2 hours. A solution of 2.96 g (18 mmol) of 3-(benzyloxy)propanal (J Org Chem, 47(27), 5400 (1982)) in 5 mL of THF was then added dropwise, and after 2 h at this temperature, the cooling bath was removed and 75 mL of 2N aqueous hydrochloric acid and 200 mL of dichloromethane were added. The organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated.

Yield: 1.3 g (16.6%)

MS (ESIpos): m/z=524 [M+H]+

¹H NMR (600 MHz, CHLOROFORM-d) δ ppm 1.41-1.47 (m, 27H) 1.70-2.20 (m, 4H) 2.40-2.53 (m, 1H) 3.11-3.38 (m, 1H), 3.59-3.78 (m, 2H) 3.91-4.01 (m, 1H) 4.13-4.20 (m, 1H), 4.52 (d, 2H) 4.93-5.09 (m, 1H), 7.28-7.37 (m, 5H)

### b) Di-tert-butyl (4S)-2-{3-(benzyloxy)-1-[(methylsulfonyl)oxy]propyl}-4-[(tert-butoxycarbonyl)amino]pentanedioate

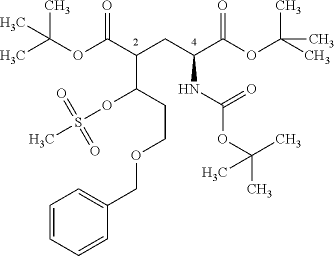 [SMILES: CC(C)(C)OC(=O)N[C@@H](CC(C(=O)OC(C)(C)C)C(CCOCc1ccccc1)OS(C)(=O)=O)C(=O)OC(C)(C)C]

4.19 g (8 mmol) of di-tert-butyl (4S)-2-[3-(benzyloxy)-1-hydroxypropyl]-4-[(tert-butoxycarbonyl)amino]pentanedioate (10a) were dissolved in 120 mL of dichloromethane and cooled to 0° C. in an ice bath. 4.05 g (40 mmol) of triethyl amine and 1.83 g (16 mmol) of methanesulfonyl chloride were added and the mixture was stirred for 2 hours at this temperature and overnight at room temperature. The reaction mixture was concentrated in vacuo and the residue was chromatographed on silica gel using a dichloromethane/ethyl acetate gradient, and the appropriate fractions were combined and concentrated.

Yield: 3.3 g (68.6%)

MS (ESIpos): m/z=602 [M+H]⁺

¹H NMR (600 MHz, CHLOROFORM-d) δ ppm 1.42-1.47 (m, 27H) 1.64-2.09 (m, 4H) 2.80-2.83 (m, 1H) 2.99 (s, 3H), 3.57-3.61 (m, 2H), 4.46-4.56 (m, 2H) 4.91-5.10 (m, 1H), 5.12-5.15 (m, 1H), 5.30 (m, 1H), 7.29-7.35 (m, 5H)

### c) Di-tert-butyl (2S) 4-[3-(benzyloxy)propylidene]-N-(tert-butoxycarbonyl) -glutamate

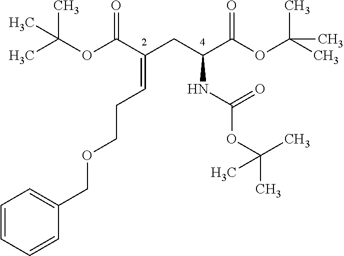 [SMILES: CC(C)(C)OC(=O)N[C@@H](C/C(=C/CCOCc1ccccc1)C(=O)OC(C)(C)C)C(=O)OC(C)(C)C]

6.02 g (10 mmol) of di-tert-butyl (4S)-2-{3-(benzyloxy)-1-[(methyl-sulfonyl)oxy]propyl}-4-[(tert-butoxycarbonyl)amino]pentanedioate (10b) were dissolved in 75 mL of dichloromethane, 3.35 mL (24 mmol) of triethylamine were added and irradiated in a microwave oven at 120° C. for 2 h. The reaction mixture was concentrated in vacuo and the residue was chromatographed on silica gel using a dichloromethane/ethyl acetate gradient and the appropriate fractions were combined and concentrated.

Yield: 3.3 g (65.3%)

MS (ESIpos): m/z=506 [M+H]⁺

¹H NMR (600 MHz, CHLOROFORM-d) δ ppm 1.38-1.51 (m, 27H), 1.73-2.02 (m, 3H) 2.37-2.90 (m, 1H), 3.20-3.40 (m, 1H), 3.48-3.61 (m, 2H), 4.10-4.44 (m, 2H) 4.48 (m, 2H), 7.33-7.36 (m, 5H)

### d) Di-tert-butyl (2S,4R)-4-[3-(benzyloxy)propyl]-N-(tert-butoxycarbonyl)-glutamate

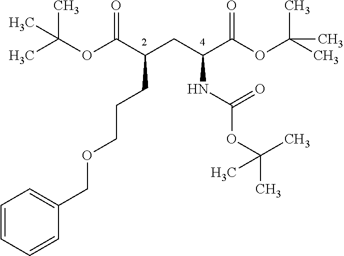 [SMILES: CC(C)(C)OC(=O)N[C@@H](C[C@@H](CCCOCc1ccccc1)C(=O)OC(C)(C)C)C(=O)OC(C)(C)C]

250 mg (0.5 mmol) of di-tert-butyl (2S) 4-[3-(benzyloxy)propylidene]-N-(tert-butoxycarbonyl)-glutamate (10c) were dissolved in 10 mL of methanol and 40 microL (0.5 mmol) of pyridine. After addition of 50 mg of palladium on charcoal (10%) under nitrogen the mixture was hydrogenated overnight at room temperature. The catalyst was then filtered off and the filtrate was concentrated in vacuo. The crude product obtained in this manner was chromatographed on silica gel using a hexane/ethyl acetate gradient and the fractions containing the diastereomeric mixture of (4R) and (4S) isomers of the title compound were combined and concentrated. This mixture was chromatographed on silica gel using a dichloromethane/ethyl acetate gradient and the appropriate fractions (R_{f} value (dichloromethane/ethyl acetate 19:1)=0.40) were combined and concentrated.

Yield: 70 mg (27.5%)

MS (ESIpos): m/z=508 [M+H]⁺

¹H NMR (600 MHz, CHLOROFORM-d) δ ppm 1.42-1.48 (m, 27H) 1.58-1.85 (m, 3H) 2.09-2.17 (m, 1H), 2.30-2.39 (m, 1H), 3.45 (m, 2H), 4.18-4.24 (m, 1H) 4.49 (s, 2H), 4.95-5.01 (m, 1H), 7.27-7.37 (m, 5H)

### e) Di-tert-butyl (2S,4R)—N-(tert-butoxycarbonyl)-4-(3-{[(4-nitrophenyl)-sulfonyl]oxy}propyl)-glutamate

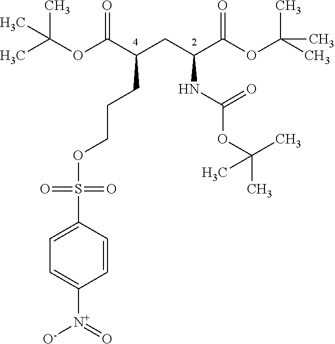 [SMILES: CC(C)(C)OC(=O)N[C@@H](C[C@@H](CCCOS(=O)(=O)c1ccc([N+](=O)[O-])cc1)C(=O)OC(C)(C)C)C(=O)OC(C)(C)C]

150 mg (0.295 mmol) of di-tert-butyl (2S,4R)-4-[3-(benzyloxy)propyl]-N-(tert-butoxycarbonyl)-glutamate (10d) were dissolved in 10 mL of methanol. 0.1 g of palladium on charcoal (15%) was added under nitrogen and the heterogeneous mixture was hydrogenated at room temperature overnight. The catalyst was then filtered off and the filtrate was concentrated in vacuo. Di-tert-butyl (2S,4R)—N-(tert-butoxycarbonyl)-4-(3-hydroxypropyl)-glutamate was identified by mass spectrometry (m/z=418 [M+H]⁺) and without further purification, the residue (120 mg) was dissolved in 7.5 mL of dichloromethane and cooled in an ice-bath. After addition of 0.17 g (168 mmol) of triethylamine and 124 mg (0.56 mmol) nitrophenylsulfonyl chloride, the mixture was stirred on ice for 4 h, overnight at room temperature and then concentrated. The crude product obtained in this manner was chromatographed on silica gel using a hexane/ethyl acetate gradient and the appropriate fractions were combined and concentrated.

Yield: 105 mg (62.2%)

MS (ESIpos): m/z=603 [M+H]⁺

¹H NMR (600 MHz, CHLOROFORM-d) δ ppm 1.43-1.46 (m, 27H), 1.61-1.73 (m, 6H), 2.06-2.10 (m, 1H), 2.32-2.34 (m, 1H), 4.11-4.15 (m, 3H), 4.95 (m, 1H), 8.10-8.12 (d, 2H), 8.40-8.43 (d, 2H)

### Example 13

### One-pot synthesis of (2S,4R)-4-(3-[¹⁸]fluoropropyl)-glutamic acid starting from Di-tert-butyl (2S,4R)—N-(tert-butoxycarbonyl)-4-(3{[(4-nitrophenyl)sulfonyl]oxy}propyl)-glutamate using a “GE tracerlab FX” synthesizer (FIG. 6)

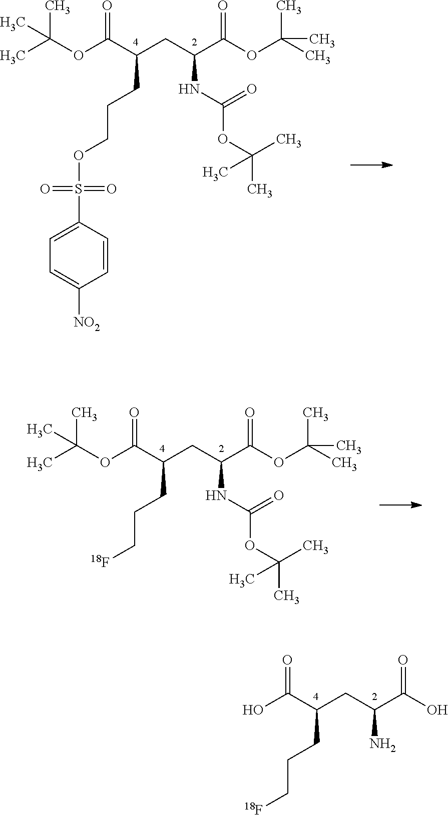

[F-18]fluoride (9.1 GBq) was trapped on a QMA cartridge (Waters, SepPak light). The activity was eluted with 1.5 mL kryptofix/potassium carbonate solution (5 mg kryptofix, 1 mg potassium carbonate in 1.25 mL acetonitrile and 0.25 mL water) into the reaction vessel. The mixture was dried (120° C., nitrogen stream, vacuum). Drying was repeated after addition of 1 ml acetonitrile. 5 mg nosylate precursor in 1 mL acetonitrile were added to the dried residue and the resulting solution was stirred at 60° C. for 1 min. 2N HCl (2 mL) was added without previous evaporation and the mixture was heated at 110° C. for 5 min. After cooling to 60° C., the solution was diluted with water (50 mL) and passed through a HR-P (Chromafix HR-P, Macherey-Nagel) and two MCX plus cartridges (Waters). The MCX cartridge was washed with saline (10 mL) and (2S,4R)-4-{3-[F-18]fluoropropyl}-glutamic acid was eluted with 10 mL formulation buffer (70 mg Na₂HPO₄2H₂O, 60 mg NaCl in 10 mL water) into the product vial.

Radiochemical yield: 4.05 GBq (63% d.c.)

Overall process time: 51 min

Radiochemical purity: >98% (determined by TLC)>
98% (determined by HPLC)

Diastereomeric ratio: >98/2 (determined by HPLC)

### Example 14

### One-pot synthesis of (2S,4R)-4-(3-[¹⁸F]fluorohexyl)-glutamic acid starting from Di-methyl (2S,4R)—N-(tert-butoxycarbonyl)-4-(6-iodohexyl)-glutamate

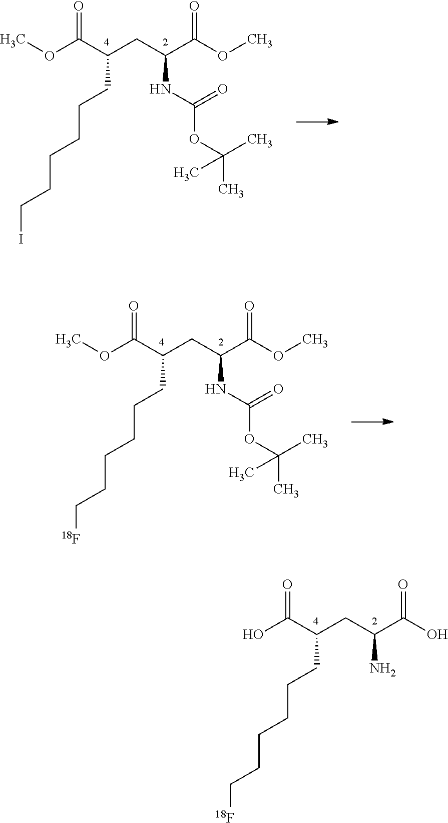 [SMILES: COC(=O)[C@@H](CCCCCCI)C[C@H](NC(=O)OC(C)(C)C)C(=O)OC ; COC(=O)[C@@H](CCCCCC[18F])C[C@H](NC(=O)OC(C)(C)C)C(=O)OC ; N[C@@H](C[C@H](CCCCCC[18F])C(=O)O)C(=O)O]

[F-18]fluoride (1.5 GBq) was trapped on a QMA cartridge (Waters, SepPak light). The activity was eluted with 1.5 mL kryptofix/potassium carbonate solution (5 mg kryptofix, 1 mg potassium carbonate in 1.25 mL acetonitrile and 0.25 mL water) into the reaction vial. The mixture was dried (120° C., nitrogen stream). Drying was repeated after addition of 1 ml acetonitrile. 5 mg iodo precursor in 1 mL acetonitrile were added to the dried residue and the resulting solution was stirred at 110° C. for 10 min. The mixture dried under gentle nitrogen stream at 110° C. After cooling to 60° C., 4M HCl (2 mL) was added and the mixture was to stirred at 150° C. for 10 min. After cooling to 60° C., the solution was diluted with water (50 mL) and passed through a OASIS HLB cartridge (HLB plus, Waters). The cartridge was washed with 2M HCl (10 mL) and water (10 mL). 422 MBq (41% d.d.) (2S,4S)-4-{6-[F-18]fluorohexyl}-glutamic acid were eluted with 5 mL formulation buffer into the product vial.

The radiochemical purity was determined by Derivatization-HPLC to be 93% (FIG. 12).

1. A method for producing compound of Formula I 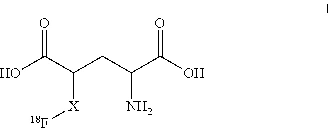
comprising the steps of:
Step 1: Synthesizing radiolabeled compound of Formula III by reacting compound of Formula II with a F-18 fluorinating agent, 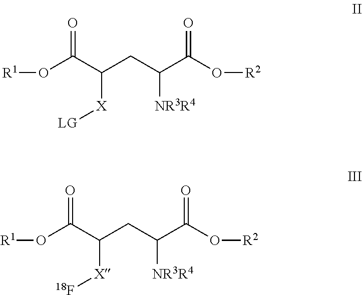
Step 2: Cleavage of the protecting groups of compound of Formula III to obtain compound of Formula I,
Step 3: Purification and formulation of compound of Formula I
wherein:
X is selected from the group comprising
a) bond,
b) branched or non-branched (C2-C10)alkyl,
c) branched or non-branched (C2-C10)alkoxy,
d) branched or non-branched (C3-C10)alkenyl,
e) branched or non-branched (C3-C10)alkynyl,
f) O—[(CH₂)ₙ—O]ₘ—(CH₂)ₒ, and
g) O—[(CH₂)ₙ—O]ₘ—(CH₂)ₒ;
n=2-6, preferably n=2 or 3;
m=1-3, preferably m=1 or 2;
o=2-6, preferably o=2 or 3;
R¹ and R² are carboxyl-protecting groups and wherein carboxyl-protecting group is independently from each other selected from
a) branched or non-branched (C₁-C₆)alkyl,
b) benzyl, and
c) allyl;
R³ and R⁴ are independently from each other selected from the group comprising:
a) hydrogen,
b) an amine-protecting group or
c) the group NR³R⁴ is a 1,3-dioxo-1,3-dihydro-2H-isoindol-2-yl (phthalimido) or an azido group; and
encompassing single isomers, tautomers, diastereomers, enantiomers, mixtures thereof and suitable salts thereof.

2. The method according to claim 1 wherein Step 3 comprises a solid-phase-extraction, preferably a cation exchange solid phase.

3. The method according to claim 1 wherein the [¹⁸F] fluorination reaction described in Step 1 is carried out at 0° C.-160° C.

4. The method according to claim 1 wherein the [¹⁸F] fluorination agent used in Step 1 is generated from a base and [¹⁸F]fluoride and the ratio of the base and compound of Formula II is greater than zero (>0) and equal or below 1 (≦1).

5. The method according to claim 1 wherein compound of Formula I has an isomeric purity of greater than 90%.

6. The method according to claim 1 wherein the method is automated and/or remote controlled.

7. The method according to claim 1 wherein compound of Formula I is (2S,4R)-4-(3-[¹⁸F]Fluoropropyl)-glutamic acid

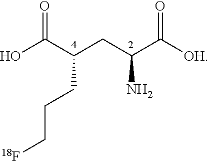 [SMILES: N[C@@H](C[C@H](CCC[18F])C(=O)O)C(=O)O]

8. The method according to claim 1 wherein compound of Formula I is (2S,4R)-4-(3-[¹⁸F]Fluoropropyl)-glutamic acid

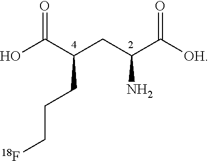 [SMILES: N[C@@H](C[C@@H](CCC[18F])C(=O)O)C(=O)O]

9. The method according to claim 1 wherein compound of Formula II is selected from the group comprising:
Di-tert-butyl (2S,4S)—N-(tert-butoxycarbonyl)-4-[3-(mesyloxy])propy]-glutamate 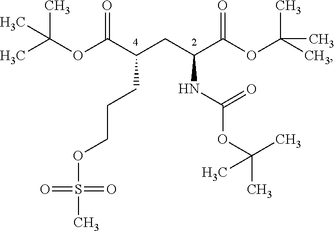 [SMILES: CC(C)(C)OC(=O)N[C@@H](C[C@H](CCCOS(C)(=O)=O)C(=O)OC(C)(C)C)C(=O)OC(C)(C)C]
Di-tert-butyl (2S,4S)—N-(tert-butoxycarbonyl)-4-[3-(tosyloxy)propy]-glutamate 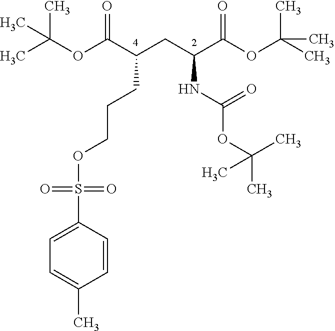 [SMILES: Cc1ccc(S(=O)(=O)OCCC[C@@H](C[C@H](NC(=O)OC(C)(C)C)C(=O)OC(C)(C)C)C(=O)OC(C)(C)C)cc1]
Di-tert-butyl (2S,4S)—N-(tert-butoxycarbonyl)-4-(3-{[(4-nitrophenyl)-sulfonyl]oxy}propyl)-glutamate 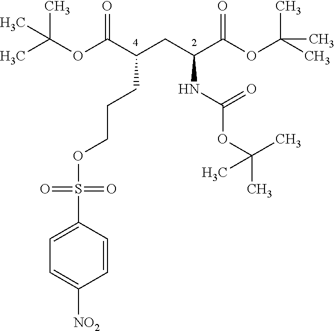 [SMILES: CC(C)(C)OC(=O)N[C@@H](C[C@H](CCCOS(=O)(=O)c1ccc([N+](=O)[O-])cc1)C(=O)OC(C)(C)C)C(=O)OC(C)(C)C]
Di-tert-butyl (2S,4R)—N-(tert-butoxycarbonyl)-4-[3-(mesyloxy]) propy]-glutamate 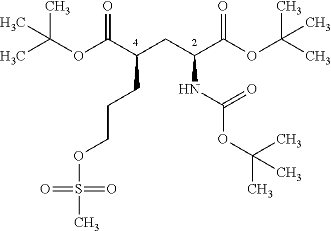 [SMILES: CC(C)(C)OC(=O)N[C@@H](C[C@@H](CCCOS(C)(=O)=O)C(=O)OC(C)(C)C)C(=O)OC(C)(C)C]
Di-tert-butyl (2S,4R)—N-(tert-butoxycarbonyl)-4-[3-(tosyloxy)propy]-glutamate 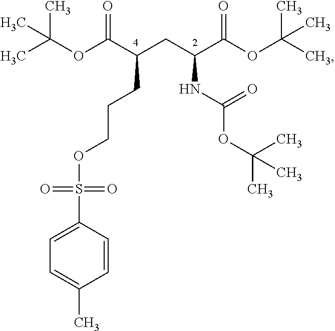 [SMILES: Cc1ccc(S(=O)(=O)OCCC[C@H](C[C@H](NC(=O)OC(C)(C)C)C(=O)OC(C)(C)C)C(=O)OC(C)(C)C)cc1]
or
Di-tert-butyl(2S,4R)—N-(tert-butoxycarbonyl)-4-(3-{[(4-nitrophenyl)-sulfonyl]oxy}propyl)-glutamate

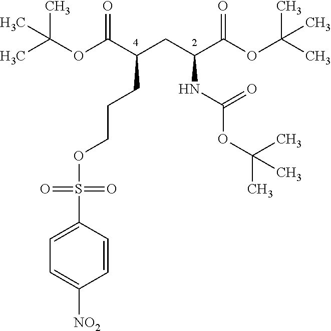 [SMILES: CC(C)(C)OC(=O)N[C@@H](C[C@@H](CCCOS(=O)(=O)c1ccc([N+](=O)[O-])cc1)C(=O)OC(C)(C)C)C(=O)OC(C)(C)C]

10. A compound of Formula IIa or IIb 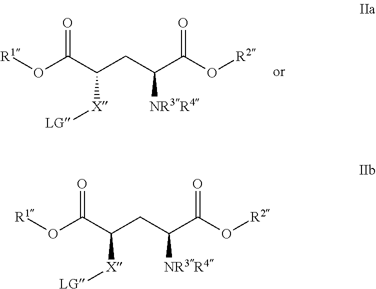
wherein:
X″ is selected from the group comprising
a) branched or non-branched (C2-C10)alkyl,
b) branched or non-branched (C2-C10)alkoxy,
c) branched or non-branched (C3-C10) alkenyl with the proviso that LG is not attached to a sp² hybridized carbon atom,
d) branched or non-branched (C3-C10) alkynyl, with the proviso that LG is not attached to asp hybridized carbon atom,
e) [(CH₂)_{n′}—O]_{m″}—(CH₂)_{o″}, and
f) O—[(CH₂)_{n″}—O]_{m″}—(CH₂)_{o″};
wherein
n″=2-6, preferably n″=2 or 3,
m″=1-3, preferably m″=1 or 2,
o″=2-6, preferably o″=2 or 3,
R^{1″} and R^{2″} are carboxyl-protecting groups and wherein carboxyl-protecting group is independently from each other selected from
a) branched or non-branched (C₁-C₆)alkyl,
b) benzyl, and
c) allyl;
R^{3″} and R^{4″} are independently from each other selected from the group comprising:
a) hydrogen,
b) an amine-protecting group or
the group NR^{3″}R^{4″} is a 1,3-dioxo-1,3-dihydro-2H-isoindol-2-yl(phthalimido) or an azido group and encompassing single isomers, tautomers, and suitable salts thereof preferably compound of Formula IIa is
Di-tert-butyl (2S,4S)—N-(tert-butoxycarbonyl)-4-[3-(mesyloxy])propy]-glutamate 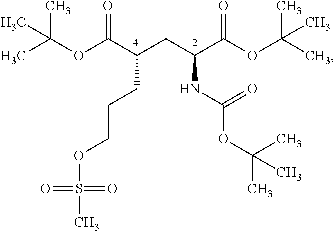 [SMILES: CC(C)(C)OC(=O)N[C@@H](C[C@H](CCCOS(C)(=O)=O)C(=O)OC(C)(C)C)C(=O)OC(C)(C)C]
Di-tert-butyl (2S,4S)—N-(tert-butoxycarbonyl)-4-[3-(tosyloxy)propy]-glutamate 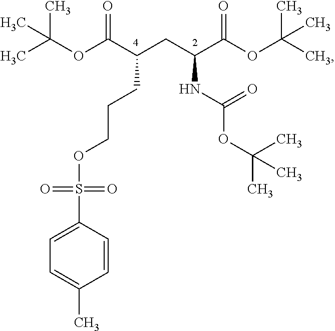 [SMILES: Cc1ccc(S(=O)(=O)OCCC[C@@H](C[C@H](NC(=O)OC(C)(C)C)C(=O)OC(C)(C)C)C(=O)OC(C)(C)C)cc1]
Di-tert-butyl (2S,4S)—N-(tert-butoxycarbonyl)-4-(3-{[(4-nitrophenyl)-sulfonyl]oxy}propyl)-glutamate 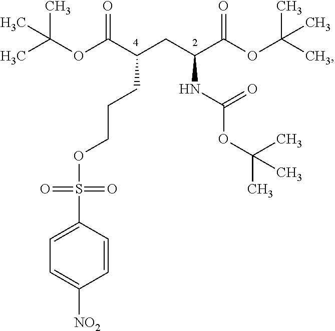 [SMILES: CC(C)(C)OC(=O)N[C@@H](C[C@H](CCCOS(=O)(=O)c1ccc([N+](=O)[O-])cc1)C(=O)OC(C)(C)C)C(=O)OC(C)(C)C]
Di-methyl (2S,4R)—N-(tert-butoxycarbonyl)-4-(6-iodohexyl)-glutamate 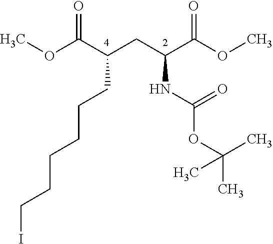 [SMILES: COC(=O)[C@@H](CCCCCCI)C[C@H](NC(=O)OC(C)(C)C)C(=O)OC]
or
preferably, compound of Formula IIb is:
Di-tert-butyl (2S,4R)—N-(tert-butoxycarbonyl)-4-[3-(mesyloxy])propyl-glutamate 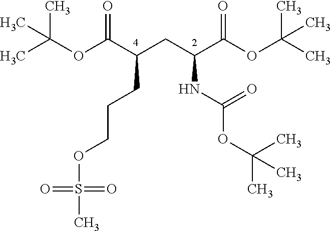 [SMILES: CC(C)(C)OC(=O)N[C@@H](C[C@@H](CCCOS(C)(=O)=O)C(=O)OC(C)(C)C)C(=O)OC(C)(C)C]
Di-tert-butyl (2S,4R)—N-(tert-butoxycarbonyl)-4-[3-(tosyloxy)propy]-glutamate 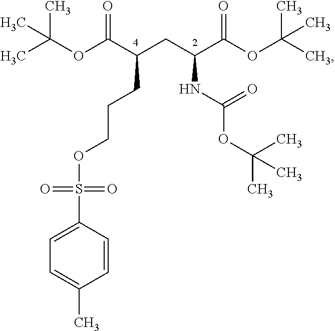 [SMILES: Cc1ccc(S(=O)(=O)OCCC[C@H](C[C@H](NC(=O)OC(C)(C)C)C(=O)OC(C)(C)C)C(=O)OC(C)(C)C)cc1]
or
Di-tert-butyl(2S,4R)—N-(tert-butoxycarbonyl)-4-(3-{[(4-nitrophenyl)-sulfonyl]oxy}propyl)-glutamate

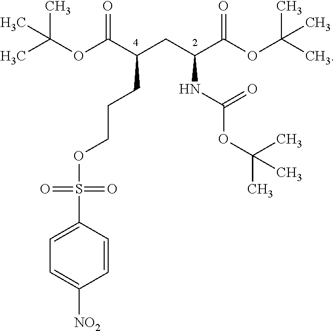 [SMILES: CC(C)(C)OC(=O)N[C@@H](C[C@@H](CCCOS(=O)(=O)c1ccc([N+](=O)[O-])cc1)C(=O)OC(C)(C)C)C(=O)OC(C)(C)C]

11. A compound of Formula Ib 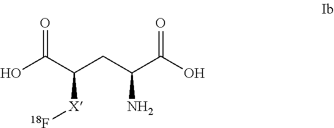
wherein:
X′ is selected from the group comprising
a) branched or non-branched (C2-C10)alkyl,
b) branched or non-branched (C2-C10)alkoxy,
c) branched or non-branched (C3-C10) alkenyl with the proviso that ¹⁸F is not attached to a sp² hybridized carbon atom,
d), branched or non-branched (C3-C10) alkynyl, with the proviso that ¹⁸F is not attached to asp hybridized carbon atom,
e) [(CH₂)_{n′}—O]_{m′}—(CH₂)_{o′}, and
f) O—[(CH₂)_{n′}—O]_{m′}—(CH₂)_{o′};
n′=2-6, preferably n′=2 or 3;
m′=1-3, preferably m′=1 or 2;
o′=2-6, preferably o′=2 or 3;
and encompassing single isomers, tautomers, diastereomers, enantiomers, mixtures thereof and suitable salts thereof,
preferably (2S,4R)-4-(3-[¹⁸F]Fluoropropyl)-glutamic acid

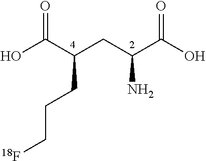 [SMILES: N[C@@H](C[C@@H](CCC[18F])C(=O)O)C(=O)O]

12. A method for obtaining a formulation comprising compound of Formula I, Formula Ia, or Formula Ib, or mixture thereof comprises the step of adding one or more physiologically acceptable vehicle or carrier, adjuvants or preservatives to a solution of compound of Formula I, Formula Ia, or Formula Ib, or mixture thereof.

13. Use of a device for carrying out the method according to claim 1 for producing compound of Formula I.

14. A Kit for producing of compound of Formula I according to claim 1 comprising
a predefined quantity of compound of Formula II according to claim 1 and
one or more solid-phase extraction cartridges/columns for the purification of compound of Formula I according to claim 1.
